SEQUENCE LISTING (4 sequences)

SEQ ID NO: 1 - DNA, 474 bp, Artificial Sequence
  Nucleotide sequence of TNF alpha chain b mutation b_tyr_119_asp
gtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatcgatctgggaggggtcttccagctggagaagggtgaccgactcagcgctgagatcaatcggcccgactatctcgactttgccgagtctgggcaggtctactttgggatcattgccctgtga

SEQ ID NO: 2 - protein, 157 aa, Artificial Sequence
  Protein sequence of TNF alpha chain b mutation b_tyr_119_asp
VRSSSRTPSDKPVAGVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPQYEPIDLGGVFQLEKGDRLSAEINRPDYLLFAESGQVYFGIIAL

SEQ ID NO: 3 - DNA, 474 bp, Artificial Sequence
  Nucleotide sequence of TNF alpha chain c mutations c_tyr_119_his, c_tyr_59_ser
gtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatcagttcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatccatctgggaggggtcttccagctggagaagggtgaccgactcagcgctgagatcaatcggcccgactatctcgactttgccgagtctgggcaggtctactttgggatcattgccctgtga

SEQ ID NO: 4 - protein, 157 aa, Artificial Sequence
  Protein sequence of TNF alpha chain c mutations c_tyr_119_his, c_tyr_59_ser
VRSSSRTPSDKPVAHVVANPQAEGQLQWLNRRANALLANGVELRDNQLVVPSEGLYLISSQVLFKGQGCPSTHVLLTHTISRIAVSYQTKVNLLSAIKSPCQRETPEGAEAKPQYEPIHLGGVFQLEKGDRLSAEINRPDYLLFAESGQVYFGIIAL

(19) United States
(12) Patent Application Publication
(10) Pub. No.: US 2004/0259155 A1
(43) Pub. Date: Dec. 23, 2004

(54) Methods of engineering spatially conserved motifs in polypeptides

(75) Inventors: John Chan, Raleigh, NC (US); Shengsheng Zhang, Framingham, MA (US); Brian Baynes, Somerville, MA (US)

Correspondence Address: ROPES & GRAY LLP, ONE INTERNATIONAL PLACE, BOSTON, MA, 02110-2624, US

(73) Assignee: Compound Therapeutics, Inc., Waltham, MA

(21) Appl. No.: 10/676,873

(22) Filed: Sep. 30, 2003

Related U.S. Application Data
(60) Provisional application No. 60/414,688, filed on Sep. 30, 2002.

(51) Int. Cl. G01N 33/53; G06F 19/00; G01N 33/48; G01N 33/50

(52) U.S. Cl. 435/7.1; 702/19

(57) ABSTRACT

The present invention provides, among other things, methods for engineering a spatially conserved motifs into a recipient polypeptide or polypeptide complex that does not naturally contain such a motif.

### RELATED APPLICATIONS

This application claims the benefit of the filing date of U.S. Provisional Application 60/414688, filed Sep. 30, 2002 and entitled “Methods of Engineering Spatially Conserved Motifs in Polypeptides”. The contents of this application, including the appendices, are incorporated by reference herein.

### BACKGROUND

Engineering proteins with new functions is currently achieved via two general methods: directed evolution and rational design. The directed evolution method applies random mutations to an initial protein framework to create numerous variants and then screen for variants that meet certain functional criteria. The directed evolution approach is very powerful for exploring unknown protein configurations but it is limited by the ability to generate and examine the immense number of possible combinations. It is particularly challenging when the desired protein properties require many concurrent changes and can not be built from incremental mutations.

The rational design method can be divided into two general approaches distinguishable by the scale of the building blocks used. One approach is to apply the basic principles of molecular physics to design novel functions by assembling amino acids, the primary unit of proteins or by assembling basic secondary peptide structures such as helices and beta-sheets into different configurations. This approach has yielded interesting results but has limited applications because our knowledge of the basic design principles is limited. A second approach is to assemble larger and more complex units of proteins with known functions but not necessarily knowing the mechanisms behind the functions. This approach is exemplified by the design of new polyketide synthentases that are capable of synthesizing chemical compounds never before seen in nature (see Carreras C. W., et al. (2000) EXS 89:89-108 and U.S. Pat. No. 6,216,816 by Chaitan Koshla).

The joining of proteins as units of known functions to create new functions is very appealing because nature, through millions of years of trials, has accumulated a large repertoire of chemical and physical properties in the form of proteins. The limitation of this method is that the functional units are spatially separated and some reactions, such as the hydrolysis of a substrate, may be performed more efficiently when the domain for binding the substrate and the domain for the hydrolysis of the substrate are in close proximity.

### SUMMARY

In part, the present invention is directed to methods of engineering spatially conserved motifs into recipient polypeptides lacking these spatially conserved motifs. In a further aspect, the present invention is directed to methods of engineering spatially conserved motifs into recipient polypeptide complexes lacking these spatially conserved motifs. In certain aspects, the invention provides engineered polypeptides and polypeptide complexes comprising a target binding functionality and an engineered spatially conserved catalytic motif.

Accordingly, in one aspect, the present invention provides a method for engineering a spatially conserved motif, e.g., a catalytic motif such as a serine protease triad or a binding motif such as a metal binding motif, into a polypeptide such as a polypeptide that binds to a ligand or substrate, e.g., an antibody, a cytokine receptor (e.g., a TNF receptor, or an interleukin receptor), or a growth factor receptor (e.g., FGF receptor). The method includes identifying a set of amino acid residues in a first polypeptide lacking the spatially conserved motif, where the set of amino acid residues have the same spatial relationship as a set of amino acid residues making up a spatially conserved motif in a second polypeptide that naturally contains the motif, and then substituting the set of residues in the first polypeptide with the set of amino acid residues making up the spatially conserved motif, such that a spatially conserved motif is engineered into a polypeptide.

In one embodiment, the set of residues that are identified in the polypeptide lacking the spatially conserved motif are less than 10 Å, 9.5 Å, 9 Å, 8.5 Å, 8.0 Å, or 7.5 Å away from a substrate binding site or a ligand binding site. In a preferred embodiment, where the set of residues comprise a serine protease catalytic motif, the serine hydroxyl moiety is less than 5.5 Å away from the peptide bond that is to be hydrolyzed.

In another embodiment, the method further includes constructing a model of the first polypeptide containing the set of substituted residues in silico and determining the existence of atomic clashes between atoms in the model. In a preferred embodiment, the model is rejected if atomic clashes are present between atoms in the model.

In another embodiment, the method further includes constructing a model of the first polypeptide containing the set of substituted residues in silico and comparing the polypeptide backbone of the first polypeptide in the presence and absence of the set of substituted residues.

In a further embodiment, the method further includes determining the root mean squared deviation of distance metrics of a polypeptide in the presence of the set of substituted residues from those in the spatially conserved motif. In one embodiment, where the set of substituted residues comprises a serine protease triad, the model is rejected if there is a root mean squared deviation of greater than 2 Å between backbone inter-α-carbon atom distances, inter-β-carbon atom distances, and hydrogen bond distances.

In a further embodiment the set of spatially conserved motif comprises 2, 3, 4, 5, 6, 7, or 8 amino acid residues, preferably 3 or 4 amino acid residues.

In another embodiment, the spatially conserved motif is a characteristic of a family of peptides, e.g., a serine protease dyad, a serine protease triad, a zinc binding motif, or an aspartic protease motif.

In another embodiment, the set of residues that are being substituted with the residues that comprise the spatially conserved motif contain at least one glycine residue.

In a further embodiment, at least one of the β carbon atoms in the set of substituted residues is positioned on the opposite side of the plane formed by the α carbons of the substituted set of residues as compared to the remaining β carbons.

In another aspect, the present invention provides a method for engineering a spatially conserved motif into a polypeptide. The polypeptide includes identifying within a family of polypeptides a set of amino acid residues that comprise a spatially conserved motif; determining the spatial relationship of the set of amino acid residues comprising the spatially conserved motif; identifying a set of amino acid residues in a polypeptide lacking the spatially conserved motif that have the same spatial relationship as the set of amino acid residues in the spatially conserved motif; and substituting the set of identified amino acid residues in the polypeptide lacking the spatially conserved motif with the set of amino acid residues that comprise the spatially conserved motif; thereby engineering a spatially conserved motif into a polypeptide.

In a further aspect, the present invention provides a method for engineering a spatially conserved motif into an oligomeric polypeptide, e.g., an oligomeric polypeptide comprising 2, 3, 4, 5, or 6 polypeptides. The method includes identifying a set of residues in an oligomeric polypeptide lacking a spatially conserved motif wherein the set of amino acid residues have the same spatial relationship as a set of residues making up a spatially conserved motif in a polypeptide that naturally contains the motif; and substituting the set of residues in the oligomeric polypeptide with the set of amino acid residues making up the spatially conserved motif; thereby engineering a spatially conserved motif into an oligomeric polypeptide.

In one embodiment, the oligomeric polypeptide has the same residues replaced on each subunit.

In another embodiment, the set of residues that are identified in the polypeptide that lacks the spatially conserved motif are less than 5 Å away from a subunit interface.

In another aspect, the invention provides a method for engineering a spatially conserved motif, e.g., a catalytic motif such as a serine protease triad or a binding motif, into a polypeptide that binds a proteinaceous substrate. The method includes identifying a set of residues in a proteinaceous substrate-bound polypeptide lacking a spatially conserved motif wherein the set of amino acids have the same spatial relationship as a set of amino acid residues making up a spatially conserved motif in polypeptide that naturally contains the motif, wherein at least one residue resides on the proteinaceous substrate; and substituting the set of residues in the proteinaceous substrate-bound polypeptide with the set of amino acid residues making up the spatially conserved motif, wherein the at least one residue residing on the proteinaceous substrate is not substituted; thereby engineering a spatially conserved motif into a polypeptide that binds a proteinaceous substrate.

In one embodiment, one of the residues that comprises the spatially conserved motif resides on the proteinaceous substrate. In the specific example where the spatially conserved motif is a serine protease triad, the histidine, aspartic acid or serine residue can be part of the proteinaceous substrate.

In another aspect, the invention provides a method of engineering a catalytic triad into a non-catalytic polypeptide. The method includes determining the spatial relationship between a set of residues that form a catalytic triad in a serine protease family of molecules; identifying a set of residues in a non-catalytic polypeptide having the same spatial relationship as the set of residues that form the catalytic triad in the serine protease family; substituting the set of identified residues in the non-catalytic polypeptide in silico with the residues that form the catalytic triad of the serine protease family; determining if there are steric clashes in the resulting molecule; and substituting the set of identified residues in the non-catalytic polypeptide with the set of residues that form the catalytic triad; thereby engineering a catalytic triad into a non-catalytic polypeptide.

Other features and advantages of the invention will be apparent from the following detailed description and claims.

### BRIEF DESCRIPTION OF THE DRAWINGS

FIG. 1 depicts a schematic representation of the protein engineering software that is employed in the methods of the invention.

FIG. 2 depicts a schematic representation of the process for engineering a spatially conserved motif into a polypeptide.

FIG. 3 depicts sequences for a TNF-alpha trimer engineered with a serine protease catalytic triad to enable cleavage of a TNF-alpha monomer.

### DETAILED DESCRIPTION

1. Overview

The present invention provides methods for engineering a spatially conserved motif into a polypeptide or polypeptide complex that does not naturally contain the motif, and polypeptides and polypeptide complexes generated by such methods. The polypeptide or polypeptide into which the spatially conserved motif is to be engineered is termed the “recipient” polypeptide or recipient polypeptide complex. Methods disclosed herein may be used, for example, to engineer a spatially conserved catalytic motif into a recipient polypeptide or polypeptide complex that binds a desired target, such that the engineered polypeptide or complex is designed to bind and catalytically modify the target. Likewise, a spatially conserved target binding motif may be engineered into a recipient polypeptide or polypeptide complex such that the engineered polypeptide or complex is designed to bind and catalytically modify the target. A target may be essentially any molecule, complex or other substance that it is desirable to affect, and preferably a target is selected such that affecting the target achieves (or is expected to achieve) a desirable therapeutic effect. Examples of desirable targets are described below.

A “spatially conserved motif,” as used herein, includes a three-dimensional arrangement of amino acid residues that is found in one or more proteins in nature, preferably in a family of proteins in nature. The spatially conserved motif typically confers upon the protein in which it is found a specific function, e.g., the ability to catalyze a reaction or the ability to bind a ligand. The three dimensional arrangement conforms to or is characterized by a number of parameters, such as distance between amino acid residues, angles between amino acid residues and the orientation of each amino acid residue in the protein. Examples of spatially conserved motifs include, but are not limited to, catalytic motifs and binding motifs. In specific embodiments, the spatially conserved motif may contain 2, 3, 4, 5, 6, 7 or 8 amino acid residues, preferably 3 or 4 amino acid residues. Examples of spatially conserved motifs with two residues include the serine protease dyad. Examples of spatially conserved motifs with three residues include the serine protease triad and the zinc binding site (Vallee, et al. (1990) Proc. Natl. Acad. Sci. USA 87:220-24). Examples of spatially conserved motifs with five residues include the mandelate racemase motif. Examples of spatially conserved motifs with six residues include the aspartic protease motif. Further examples of spatially conserved motifs can be found on the PROCAT webpage maintained by the University College, London. This webpage can be found at http://www.biochem.ucl.ac.uk/bsm/PROCAT/PROCAT. html. Another example is the E.C.2.1.2.2 Phosphoribosylglycinamide formyltransferase, with an active site consensus template: His 108-Asp 144-Asn 106 and the following table of residues geometries derived from several family members:

| Atom | Alt. Conf. | Residue | Number | x | y | z |
|---|---|---|---|---|---|---|
| CB | — | His | 108 | −1.509 | 0.024 | 0.016 |
| CG | — | His | 108 | 0.000 | 0.000 | 0.000 |
| ND1 | — | His | 108 | 0.784 | −1.075 | 0.000 |
| CD2 | — | His | 108 | 0.816 | 1.120 | 0.000 |
| CE1 | — | His | 108 | 2.034 | −0.671 | 0.006 |
| NE2 | — | His | 108 | 2.049 | 0.654 | 0.006 |
| CG | — | Asp | 144 | 5.587 | 0.103 | −1.107 |
| OD1 | — | Asp | 144 | 5.424 | 0.158 | −2.012 |
| OD2 | — | Asp | 144 | 4.931 | −0.593 | −0.794 |
| CG | — | Asn | 106 | 2.953 | −2.797 | −3.603 |
| OD1 | — | Asn | 106 | 3.410 | −2.260 | −2.651 |
| ND2 | — | Asn | 106 | 2.269 | −3.915 | −3.419 |

An additional example is the E.C.3.1.1.7 Acetylcholinesterase, active site consensus template: Ser 58-His 440-Glu 327, with spatial geometries shown below:

| Atom | Alt. Conf. | Residue | Number | x | y | z |
|---|---|---|---|---|---|---|
| OG | — | Ser | 58 | 4.880 | 1.351 | 0.277 |
| CB | — | His | 440 | −1.499 | −0.039 | −0.046 |
| CG | — | His | 440 | 0.000 | 0.000 | 0.000 |
| ND1 | — | His | 440 | 0.809 | −1.090 | 0.000 |
| CD2 | — | His | 440 | 0.818 | 1.102 | 0.000 |
| CE1 | — | His | 440 | 2.069 | −0.675 | −0.003 |
| NE2 | — | His | 440 | 2.094 | 0.649 | −0.006 |
| OE1 | — | Glu | 327 | −0.625 | −3.366 | 0.340 |

An additional example is the E.C.3.1.1.4 Phospholipase A2, active site consensus template: Ca 1- His 48-Asp 99:

| Atom | Alt. Conf. | Residue | Number | x | y | z |
|---|---|---|---|---|---|---|
| CA | — | Ca | 1 | −1.488 | −7.079 | −0.744 |
| CB | — | His | 48 | −1.514 | −0.062 | 0.015 |
| CG | — | His | 48 | 0.000 | 0.000 | 0.000 |
| ND1 | — | His | 48 | 0.813 | −1.100 | 0.000 |
| CD2 | — | His | 48 | 0.810 | 1.095 | 0.000 |
| CE1 | — | His | 48 | 2.064 | −0.679 | 0.013 |
| NE2 | — | His | 48 | 2.086 | 0.638 | 0.022 |
| OD2 | — | Asp | 99 | 4.471 | 2.081 | −0.066 |

An additional example is the E.C.3.2.1.4 Cellulase, active site consensus template: Asp 265-Asp 117:

| Atom | Alt. Conf. | Residue | Number | x | y | z |
|---|---|---|---|---|---|---|
| CG | — | Asp | 265 | 0.000 | 0.000 | 0.000 |
| OD1 | — | Asp | 265 | 0.601 | 1.071 | 0.000 |
| OD2 | — | Asp | 265 | 0.600 | −1.069 | 0.000 |
| CG | — | Asp | 117 | 3.789 | 6.092 | −8.849 |
| OD1 | — | Asp | 117 | 4.661 | 5.500 | −9.436 |
| OD2 | — | Asp | 117 | 3.439 | 5.762 | −7.728 |

An additional example is the E.C.3.2.1.1 Alpha-amylase, active site consensus template: Glu 204-Asp 179:

| Atom | Alt. Conf. | Residue | Number | x | y | z |
|---|---|---|---|---|---|---|
| CG | — | Glu | 204 | −1.501 | 0.058 | 0.015 |
| CD | — | Glu | 204 | 0.000 | 0.000 | 0.000 |
| OE1 | — | Glu | 204 | 0.592 | −1.082 | 0.000 |
| OE2 | — | Glu | 204 | 0.596 | 1.089 | 0.000 |
| CG | — | Asp | 179 | 2.484 | −3.526 | 2.743 |
| OD1 | — | Asp | 179 | 2.368 | −2.303 | 2.789 |
| OD2 | — | Asp | 179 | 3.440 | −4.120 | 2.257 |

An additional example is the E.C.3.4.17.1 Carboxypeptidase A, active site consensus template: Zn 308-Glu 270-Arg 127:

| Atom | Alt. Conf. | Residue | Number | x | y | z |
|---|---|---|---|---|---|---|
| Zn | — | Zn | 308 | 5.395 | −0.411 | −1.218 |
| CG | — | Glu | 270 | −1.506 | −0.019 | −0.025 |
| CD | — | Glu | 270 | 0.000 | 0.000 | 0.000 |
| OE1 | — | Glu | 270 | 0.607 | −1.100 | 0.000 |
| OE2 | — | Glu | 270 | 0.597 | 1.082 | 0.000 |
| CZ | — | Arg | 127 | 8.859 | 0.555 | 2.451 |
| NH1 | — | Arg | 127 | 9.970 | 0.063 | 2.372 |
| NH2 | — | Arg | 127 | 8.519 | 0.239 | 2.454 |

An additional example is the E.C.3.4.23.15 Renin, aspartic proteinase active site consensus template: Thr 34-Gly 35-Asp 33-Asp 213-Thr 214-Gly 215:

| Atom | Alt. Conf. | Residue | Number | x | y | z |
|---|---|---|---|---|---|---|
| OG1 | — | Thr | 34 | −0.208 | 5.701 | −1.591 |
| N | — | Gly | 35 | 1.080 | 2.686 | −2.354 |
| CB | — | Asp | 33 | −1.511 | −0.030 | −0.003 |
| CG | — | Asp | 33 | 0.000 | 0.000 | 0.000 |
| OD2 | — | Asp | 33 | 0.610 | −1.076 | 0.000 |
| OD1 | — | Asp | 33 | 0.612 | 1.080 | 0.000 |
| CB | — | Asp | 213 | 4.005 | 4.919 | 0.359 |
| CG | — | Asp | 213 | 3.760 | 3.463 | 0.533 |
| OD1 | — | Asp | 213 | 2.651 | 2.991 | 0.481 |
| OD2 | — | Asp | 213 | 4.702 | 2.783 | 0.725 |
| OG1 | — | Thr | 214 | −2.088 | 4.493 | 1.007 |
| N | — | Gly | 215 | 0.535 | 3.119 | 2.541 |

An additional example is the E.C.3.8.1.5 Haloalkane dehalogenase, active site consensus template: Asp 124-His 289-Asp 260:

| Atom | Alt. Conf. | Residue | Number | x | y | z |
|---|---|---|---|---|---|---|
| OD1 | — | Asp | 124 | 4.527 | 1.106 | −0.055 |
| CB | — | His | 289 | −1.491 | −0.116 | −0.036 |
| CG | — | His | 289 | 0.000 | 0.000 | 0.000 |
| ND1 | — | His | 289 | 0.813 | −1.110 | 0.000 |
| CD2 | — | His | 289 | 0.800 | 1.092 | 0.000 |
| CE1 | — | His | 289 | 2.080 | −0.696 | 0.000 |
| NE2 | — | His | 289 | 2.089 | 0.628 | 0.004 |
| OD2 | — | Asp | 260 | −0.362 | −3.652 | 0.065 |

An additional example is the E.C.6.1.1.18 Glutamine—tRNA ligase, active site consensus template: His 43-Asn 36-Lys 270:

| Atom | Alt. Conf. | Residue | Number | x | y | z |
|---|---|---|---|---|---|---|
| CB | — | His | 43 | −1.503 | −0.008 | −0.006 |
| CG | — | His | 43 | 0.000 | 0.000 | 0.000 |
| ND1 | — | His | 43 | 0.765 | −1.083 | 0.000 |
| CD2 | — | His | 43 | 0.789 | 1.117 | 0.000 |
| CE1 | — | His | 43 | 2.008 | −0.661 | 0.005 |
| NE2 | — | His | 43 | 2.002 | 0.650 | 0.004 |
| CG | — | Asn | 36 | 9.048 | −1.244 | −3.077 |
| OD1 | — | Asn | 36 | 7.946 | −0.750 | −3.053 |
| ND2 | — | Asn | 36 | 9.798 | −1.235 | −1.986 |
| NZ | — | Lys | 270 | 5.451 | −0.595 | −1.919 |

An additional example is the E.C.3.4.22.1 Cathepsin B, Cys-His-Asn protease active site consensus template: Cys 29-His 199-Asn 219:

| Atom | Alt. Conf. | Residue | Number | x | y | z |
|---|---|---|---|---|---|---|
| SG | — | Cys | 29 | 0.509 | −4.008 | 2.034 |
| CB | — | His | 199 | −1.503 | −0.083 | 0.011 |
| CG | — | His | 199 | 0.000 | 0.000 | 0.000 |
| ND1 | — | His | 199 | 0.820 | −1.104 | 0.000 |
| CD2 | — | His | 199 | 0.810 | 1.090 | 0.000 |
| CE1 | — | His | 199 | 2.092 | −0.698 | 0.007 |
| NE2 | — | His | 199 | 2.097 | 0.630 | −0.002 |
| OD1 | — | Asn | 219 | 4.356 | 2.205 | 0.392 |

An additional example is the E.C.3.4.24.23 Matrilysin, metalloproteinase active site consensus template: Zn 1-Glu 219-Ala 182:

| Atom | Alt. Conf. | Residue | Number | x | y | z |
|---|---|---|---|---|---|---|
| ZN | — | Zn | 1 | 4.285 | −0.419 | −2.487 |
| CG | — | Glu | 219 | −1.519 | 0.050 | 0.025 |
| CD | — | Glu | 219 | 0.000 | 0.000 | 0.000 |
| OE1 | — | Glu | 219 | 0.613 | −1.081 | 0.000 |
| OE2 | — | Glu | 219 | 0.611 | 1.078 | 0.000 |
| O | — | Ala | 182 | 2.397 | 0.946 | 3.119 |

Further information regarding any of the preceding categories may be found in the Procat database.

In certain embodiments, the methods include identifying a set of amino acid residues in a recipient polypeptide or polypeptide complex. The set of amino acid residues is selected so as to have a spatial relationship that is similar to that of a set of amino acid residues making up a spatially conserved motif in a second polypeptide that naturally contains the motif. Whether the set of amino acid residues is a suitable “match” for the spatially conserved motif may be determined for reasons related to a theoretical or empirical understanding of the properties of the spatially conserved motif; the threshold for matching may also be set for practical reasons more or less unrelated to the properties of the spatially conserved motif. For example, the threshold for identifying a set of amino acids in the recipient polypeptide or polypeptide complex may be based upon the expected flexibility of the motif. Some motifs may be tolerant of significant deviation from a precise geometry, allowing a lower threshold for matching, while other motifs may be relatively intolerant, leading to a higher threshold for matching. The expected flexibility of a motif may be determined by aligning a plurality of such motifs and assessing the variation in the geometry. As another example, whether a set of amino acid residues is a suitable match may be determined by setting a threshold based on the number of engineered variants that are to be constructed and tested. If one wishes to test a broad array of engineered proteins, a low threshold for matching may be set. If one wishes to test few engineered proteins, a higher threshold may be set. It is also possible to test a variety of thresholds to identify different sets of amino acids in the recipient polypeptide or complex, and the threshold providing the most desirable results can be adopted. In preferred embodiments, the set of amino acid residues identified in the recipient polypeptide or polypeptide complex has a spatial relationship that exactly (to the level of accuracy used in modeling) matches the spatial relationship of the conserved motif. The set of residues in the recipient polypeptide or polypeptide complex may be substituted with the set of amino acid residues making up the spatially conserved motif, such that a spatially conserved motif is engineered into the recipient. In certain embodiments, additional residues that participate flexibly in active site function may be included in the modeled structures. This approach takes into account the tendency of some more distant residues that are not traditionally recognized as part of an active site to participate in reactions catalyzed by the active. Often such residues will be dispensable, but including these residues may improve the activity of an engineered motif.

2. Definitions

For convenience, certain terms employed in the specification, examples, and appended claims are collected here. Unless defined otherwise, all technical and scientific terms used herein have the same meaning as commonly understood by one of ordinary skill in the art to which this invention belongs.

The term “spatial relationship,” as used herein, refers to the geometric relationship between amino acid residues in a spatially conserved motif. The spatial relationship is defined by, for example, the distance between amino acid residues, the angle between amino acid residues, and the orientation of the amino acid residues.

The term “amino acid residue” as used herein, refers to naturally and non-naturally occurring amino acid residues and analogs or derivatives thereof.

The term “model,” as used herein, includes a model generated by a computer using the coordinates of a protein as determined by, for example, crystallography or nuclear magnetic resonance. The term “model” further includes a model of a polypeptide whose structure is unknown that is generated, for example, using a technology known as threading. Models generated using the methods of the invention can have a substrate, a ligand, or a cofactor bound to the polypeptide of interest or can have a substrate, a ligand, or a cofactor modeled into the binding site of the polypeptide of interest based on the binding of similar molecules to homologous polypeptides.

As used herein the term “polypeptide backbone” refers to the repeating unit structure that in inherent in every polypeptide. The polypeptide backbone comprises amide bonds that connect amino acids and make up the repeating unit of amide nitrogen, amino acid α-carbon, and carbonyl carbon.

The term “proteinaceous substrate” as used herein, refers to a molecule comprising a protein or a protein-like moiety. Thus, the term encompasses molecules that are naturally occurring proteins, fragments of naturally occurring proteins, and engineered polypeptides comprising naturally occurring amino acids, analogs of naturally occurring amino acids, and combinations thereof.

The term “root mean squared deviation,” as used herein, is a statistical term that allows for comparison of the difference of the positions of the atoms in two different polypeptide structures. Technically, the root mean squared deviation (RMSD) is the square root of the average value of the square of the individual deviations from the mean.

The term “serine protease triad” as used herein, refers to a catalytic motif found in many proteases which comprises the three residues: serine, aspartic acid and histidine. The catalytic mechanism of a serine protease triad involves two distinct steps: an acylation step in which the peptide bond is cleaved and an ester linkage is formed and a de-acylation step in which the ester is hydrolyzed and the enzyme containing the triad is regenerated.

The term “set of amino acid residues” as used herein refers to at least 2, 3, 4, 5, 6, 7, 8 or more amino acid residues. The amino acid residues can be naturally and non-naturally occurring amino acids, and analogues or derivatives thereof.

The term “substrate binding site,” as used herein, refers to the region of a polypeptide where a substrate binds. For purposes of the present invention, a substrate can be a molecule that is altered, e.g., physically or chemically altered, by a polypeptide once it binds to the polypeptide, or a substrate can be a molecule that binds to a polypeptide without being altered.

The term “ligand binding site,” as used herein, refers to the region of a polypeptide through which the polypeptide interacts with a ligand. The ligand binding site, comprises the residues that physically interact with the ligand as well as the residues that form the docking surface surrounding these residues.

Various additional aspects of the methods of the invention are described in detail in the following sections.

3. Spatially Conserved Motifs

Certain methods of the invention pertain to engineering a spatially conserved motif found in a polypeptide or family of polypeptides, into a polypeptide that is lacking this motif. Further, the spatial relationship between the amino acid residues that comprise the spatially conserved motif are maintained when substituted into the polypeptide that naturally lacks the motif. Spatially conserved motifs may also be identified in protein complexes (e.g., in the binding interface of an antibody).

A spatially conserved motif may be essentially any recognizable motif that confers a desired functionality. For example, a spatially conserved motif may be a binding motif or a catalytic motif. The term “binding motif,” as used herein, includes a set of amino acid residues that allow a polypeptide to interact with another molecule, such as a polypeptide, a metal or a DNA sequence. The binding motif comprises amino acid residues that have a specific geometric shape inherent to the motif. This geometric shape encompasses distance between the amino acid residues, angles between amino acid residues and the orientation of the amino acid residues. Examples of binding motifs include metal binding motifs, e.g., zinc, iron or copper motifs, leucine zipper binding motifs and DNA binding domains. As used herein, the term “catalytic motif” includes a set of amino acid residues which, when assembled in the correct geometric pattern and correct orientation, are capable of catalyzing a reaction, e.g., proteolysis, hydrolysis, phosphorylation, myristylation, glycosylation, or amidation.

In certain preferred embodiments, a binding motif is selected for its tendency to bind to a selected target, preferably binding with a K_{D} less than 10⁻⁵, 10⁻⁶, 10⁻⁷, 10⁻⁸, 10⁻⁹ or less. In certain preferred embodiments, a catalytic motif is selected for its ability to catalyze a reaction with a selected target.

Methods disclosed herein often include obtaining the spatial relationship of the set of residues that comprise the spatially conserved motif. In one embodiment, this knowledge is available in the art or is otherwise already available (e.g., from a proprietary database) and does not need to be determined. For example, such information may be found on the PROCAT website. In another embodiment, the spatial relationship between the set of amino acid residues that comprise the spatially conserved motif is determined by evaluating a number of structures, e.g., crystal structures or nuclear magnetic resonance structures, for the presence of the motif and determining various geometric parameters, such as the distance, angle, and orientation between the amino acid residues, or particular atoms thereof, that comprise the spatially conserved motif. This can be done, for example, using a software package that has the ability to search a set of structures and determine the presence or absence of a set of conserved residues. The software package has the ability to determine the spatial relationship that exists between the set of residues in the polypeptide (or polypeptide complex) and other important areas of the polypeptide, e.g., the site where the substrate or ligand binds, or the proximity to the binding site for other molecules, e.g., cofactors or allosteric regulators.

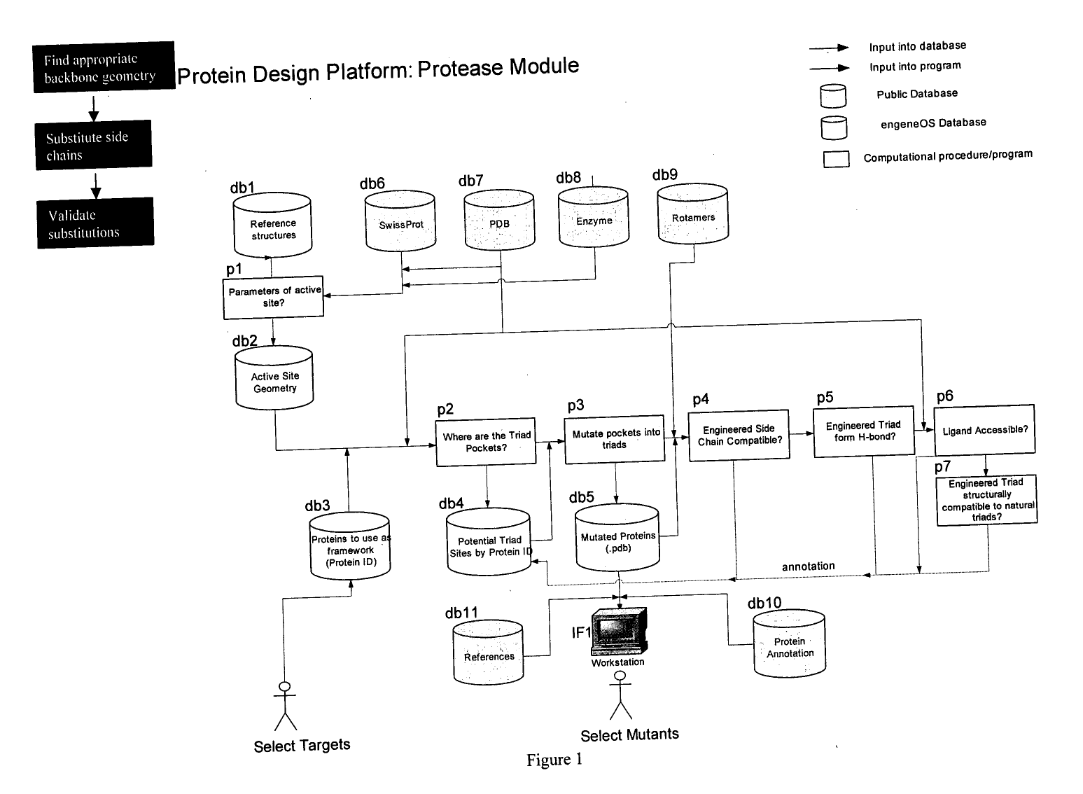

In a preferred embodiment, the software package elucidates the spatial relationship of the set of amino acid residues that comprise the spatially conserved motif in the following way (see FIG. 1):

1. Obtains a set of protein structures from the Protein Data Bank (PDB) that contain the spatially conserved motif of interest

2. Identifies the alpha- and beta-carbons of each amino acid residue that comprise the spatially conserved motif

3. Computes the distance between all distinct pairs of alpha-carbons that comprise the spatially conserved motif

4. Computes the distance between all distinct pairs of beta-carbons that comprise the spatially conserved motif

5. Computes all unique angles between all possible planes defined by sets of three alpha-carbons or beta-carbons

6. Defines the set of distances and angles computed in steps 3, 4, and 5 as the spatial relationship characteristic of the spatially conserved motif

Once the spatial relationship of the set of amino acid residues that comprise the spatially conserved motif is known or determined, a polypeptide of interest into which the spatially conserved motif will be engineered is evaluated to identify a set of amino acid residues in this polypeptide that have a spatial relationship that matches that of the set of amino acid residues making up the spatially conserved motif. The polypeptides into which the spatially conserved motif is to be engineered may be polypeptides that bind a substrate, e.g., a binding protein, a receptor, a soluble fragment of a receptor, an antibody, or fragment of an antibody.

It has been shown that the distances and overall geometry between amino acid residues in spatially conserved motifs is highly conserved (Wallace, et al. (1996) Prot. Sci. 5:1001-13; Wallace et al. (1997) Prot. Sci. 6:2308-28). Accordingly, when looking for amino acid residues to be replaced in the recipient polypeptide or polypeptide complex, it is this spatial relationship that is most important for identifying a match. Therefore, preferably there is less than 1.0 Å, 0.9 Å, 0.8 Å, 0.7 Å, 0.6 Å, 0.5 Å, 0.4 Å, 0.3 Å, 0.2 Å, 0.1 Å, 0.05 Å, or 0.025 Å deviation in the distance between residues, e.g., as measured from the α-carbon, or β-carbon, in the set of amino acid residues that are to be replaced and the distance between the amino acid residues in the set of residues that comprise the spatially conserved motif. The distance between the amino acid residues in the spatially conserved motif can vary to different degrees depending on the type of motif that is being engineered. The range of this variance is determined functionally and can be determined by evaluating a number of structures that naturally contain the spatially conserved motif to determine the variance that is present. In a further embodiment, there should be a difference of less than 30, 20, 10, 5, 4, 3, 2, 1, or 0.5 degrees between the angles created by the amino acid residues in the set of residues that are to be replaced and the angles created by the amino acid residues in the set of residues that comprise the spatially conserved motif.

The potential sites of substitution can be further analyzed by, for example, building a model of the substituted polypeptide by removing the amino acid side chains naturally present in the polypeptide and modeling in the substituted amino acid side chains. After allowing the substituted polypeptide to relax a skilled artisan can compare the root mean squared deviation of the backbone carbon atoms in the polypeptide to see if there has been any movement of the backbone to accommodate the new residues. If there is a deviation of more than, for example, 0.5 Å, 0.8 Å, 1.0 Å, 1.2 Å, 1.4 Å, 1.6 Å or 1.8 Å the model will be rejected and the actual substituted polypeptide will not be made.

In an alternate embodiment, a model is built in which the amino acid side chains are replaced with the residues from the spatially motif and the model is evaluated to determine if the introduction of the new set of residues causes steric clashes between the atoms of the resulting polypeptide. In order to evaluate the possibility of atomic clashes, a library of rotomers is used to build the model. The library contains every 5 degree rotomer of each chi angle in the amino acid side chain. If steric clashes are determined, the model will be rejected and the actual substituted polypeptide will not be made.

In one embodiment, the methods of the invention utilize glycine in addition to the remaining 19 amino acids, in the evaluation of the polypeptide into which the spatially conserved motif is to be engineered. It is important to note that glycine, which lacks a β-carbon, needs to be treated in a unique way in order for glycine residues to be used in the methods of the invention. The searching algorithms and modeling algorithm use the proton that is present in the place of the β-carbon to do the calculations, essentially substituting that proton for a carbon atom. As a result, glycine residues are not normally factored into the determination of potential substitution sites. This is a limitation of the approach of Iengar, P. et al. ((1999)Protein Engineering 12:649-55). By eliminating any combination of residues that contain a glycine residue the method of Iengar, et al. greatly limits the potential number of sites available for substitution. In contrast, the methods of the invention allow glycine residues to be factored in the determination of potential substitution sites by artificially defining the position of a β-carbon on glycine residues to be the position of the glycine hydrogen atom. The assumption allows each reside in the polypeptide to have a β-carbon, and accordingly, to be used in the algorithm that searches polypeptides for potential sites of insertion.

A further limitation of Iengar, P. et al. that is overcome by the methods of the present invention, is related to the orientation of the amino acid side chains. Iengar, P. et al. require that all of the substituted residues have their β-carbons on the same side of the plane that is created by the α-carbons. In the present invention, this constraint is removed to more accurately describe the geometries present in nature. This allows for more potential sites of insertion.

The determination of a potential set of residues in a candidate polypeptide, i.e., a polypeptide lacking the spatially conserved motif, to be replaced with the set of residues that are present in the spatially conserved motif is done using a software program. This software program automatically evaluates several criteria that are required for the spatially conserved motif to be introduced successfully into the candidate polypeptide. For example, the software program:

1. Determines the amino acid residue locations for the introduction of mutations in a polypeptide lacking the spatially conserved motif that may result in the spatially conserved motif by comparing the inter-alpha-carbon and inter-beta-carbon distances in the polypeptide lacking the spatially conserved motif with those distances in the spatially conserved motif.

2. For all of the locations discovered in step 1, performs in silico mutations to introduce the residues in the spatially conserved motif into the polypeptide lacking the spatially conserved motif.

3. For all of the mutations in step 2, determines the proximity of the atoms of the spatially conserved motif that must interact with the substrate to the nearest target atom in the substrate.

4. Determines the geometry of all possible rotamers of the spatially conserved motifs created in step 3.

5. For each of the rotamers generated in step 4, determines:

a. The likelihood that the resulting protein structure will be stable, based on the lack of steric clashes in the resulting rotamers with themselves and the remainder of the protein.

b. The similarity of the active site in the resulting protein structure to the spatially conserved motifs found in nature. In one embodiment relating to the insertion of a serine protease motif, this similarity is determined as the RMSD of the inter-alpha-carbon distances, inter-beta-carbon distances, and hydrogen bond distances of the resulting serine protease triad.

Often this analysis will determine more than one acceptable position for substitution of the set of residues that comprise the spatially conserved motif. The skilled artisan can then choose to decrease the number of acceptable positions by requiring that the criteria outlined above in steps 3 and 5 be more stringent.

Experimental substitution of residues may be done by techniques that are standard in the art of molecular biology and found, for example, in Sambrook, J., Fritsh, E. F., and Maniatis, T. (1989) Molecular Cloning: A Laboratory Manual. 2nd, ed., Cold Spring Harbor Laboratory, Cold Spring Harbor Laboratory Press, Cold Spring Harbor, N.Y.,. For example, substitution may be performed using polymerase chain reaction mediated mutagenesis. Following mutagenesis, the nucleotide sequence of the DNA encoding the substituted polypeptide can be inserted into an expression vector and the substituted polypeptide can be expressed recombinantly. Further, once the polypeptide is expressed, it can be purified and assayed or tested for a desired activity.

4. Recipients and Targets

As described herein, a spatially conserved motif may be engineered into a recipient polypeptide that has a matching set of amino acids. Applicants have further adapted this approach for use with recipient polypeptide complexes, where the residues of the spatially conserved motif may be introduced into a plurality of polypeptides that bind so as to form the spatially conserved motif. This approach is particularly advantageous in part because complexes of proteins will tend to have a larger set of possible sites for engineering.

Essentially any polypeptide or complex may be selected to be a recipient. Often a recipient will be selected for a desirable property. For example, a recipient may naturally bind to a target of interest or have catalytic activity against the target of interest. A recipient may also possess desirable pharmacokinetic properties, such as serum stability, bioavailability, low toxicity, low immunogenicity, etc.

In certain embodiments, the recipient polypeptide may be a complex, such as an oligomeric complex. For instance, the substitution of the spatially conserved residues can be done in an oligomer such that the residues that actually comprise the spatially conserved motif reside on different subunits of the oligomer. For example, a set of residues that have a catalytic function may be substituted into an oligomer such that one or two residues are substituted on each subunit and upon association of the subunits an active catalytic site is formed. In one embodiment, the oligomer is a homo-oligomer, i.e., made up of identical subunits, and the same residues are substituted in each subunit. In another embodiment, the oligmer is a homo-oligomer and different residues are substituted in one or more of the subunits to create a spatially conserved motif. Lastly, the oligomer may be a hetero-oligomer and may have different residues substituted in each subunit.

In another embodiment, one or more residues on a target ligand or substrate may comprise one or more of the residues that comprise the spatially conserved motif. For example, a spatially conserved catalytic motif comprising three amino acid residues may be substituted into a protein lacking said motif by substituting two residues on the polypeptide and one amino acid residue on the substrate. It is important to note that the residue or residues on the substrate are not being substituted, and that an existing amino acid is being used to generate the spatially conserved motif.

Preferred recipient proteins and protein complexes include proteins that already have one or more desired function(s). For example, a protein may naturally bind a ligand with high affinity and the modification of the ligand, e.g., by cleavage or phosphorylation, may be advantageous. Accordingly, this polypeptide would be a good recipient such that the resulting polypeptide has the same binding affinity for its natural ligand and a new activity, e.g., a catalytic activity capable of modifying this ligand. Examples of proteins to be used include, but are not limited to: antibodies and portions thereof; binding proteins and functional fragments thereof such as cytokine receptors and growth factor receptors; and cell surface receptors and soluble domains thereof. In certain preferred embodiments, a recipient is a soluble portion of a receptor or an antibody (or binding portion thereof) that binds to any of the targets described herein.

In certain embodiments, a ligand (or binding portion thereof) of a receptor or other cell surface molecule may be employed as a recipient and preferably engineered to contain a catalytic motif that catalyzes an alteration in the receptor. Merely to illustrate, examples of ligands suitable for use as recipients of the present invention are listed in the Table I below.

TABLE I
Ligand Recipients and Cognate Receptor Targets

| Ligand | Receptor | Cell type |
|---|---|---|
| apolipoproteins | LDL | liver hepatocytes, |
| | | vascular endothelial cells |
| insulin | insulin receptor | |
| transferrin | transferrin receptor | endothelial cells |
| Mac-1 | L selectin | neutrophils, leukocytes |
| VEGF | Flk-1, 2 | Tumor epithelial cells |
| basic FGF | FGF receptor | Tumor epithelial cells |
| EGF | EGF receptor | epithelial cells |
| VCAM-1 | a₄b₁ integrin | vascular endothelial cells |
| ICAM-1 | a_{L}b₂ integrin | vascular endothelial cells |
| PECAM-1/CD31 | aᵥb₃ integrin | vascular endothelial |
| | | cells, activated platelets |
| osteopontin | aᵥb₁ integrin | endothelial cells and |
| | aᵥb₅ integrin | smooth muscle cells in |
| | | atherosclerotic plaques |
| RGD sequences | aᵥb₃ integrin | tumor endothelial cells, |
| | | vascular smooth muscle |
| | | cells |
| HIV GP 120/41 or | CD4 | CD4⁺ lymphocytes |
| GP120 | | |

In certain embodiments, an engineered polypeptide or polypeptide complex may be designed to bind and act on a target that is a biologically active molecule (“targeted biomolecule”), e.g., including solvent accessible extracellular and intracellular substrates, as well as extracellular or cytoplasmic portions of membrane associated substrates. These include, but are not limited to, targets from among such classes as protein and peptides, nucleic acids, lipids, small molecules including extracellular factors (such as steroids and neurotransmitters) and intracellular second messengers (such as phosphorylated inositol and cAMP). By modifying the functional performance of a targeted substrate of biological relevance, the subject engineered proteins can be used to alter such cellular processes as gene expression, morphology, cell adhesion, growth, proliferation, migration, differentiation and/or viability of cell.

(a) Extracellular Targets

In certain embodiments, the target is an extracellular target, including target molecules that are typically located entirely outside of a cell and target molecules that are inserted into a cellular membrane but have a portion that is exposed to the extracellular environment. Several categories of extracellular targets are recognizable, including, for example, diffusible extracellular molecules (e.g., growth factors, serum proteins, antibodies, any diffusible small molecule, extracellular nucleotides, lipids), extracellular molecules that are part of an insoluble aggregate (e.g., β-amyloid protein, constituents of atherosclerotic plaques, insoluble fibrin fibers), membrane associated proteins and other membrane bound moieties (e.g., transmembrane proteins, lipids, membrane associated polysaccharides), and constituents of or associated with an organized extracellular matrix.

Accordingly, engineered polypeptides and complexes can be used to alter, e.g., inhibit or potentiate, such cell-surface mediated signaling as autocrine signaling (self-signaling), paracrine signaling (between nearby cells), and/or endocrine signaling (over a long distance, usually via the bloodstream or other bodily fluid). The subject engineered polypeptides or polypeptide complexes can also be used to alter juxtacrine signaling, e.g., signaling consequences of cell contact.

Various illustrative examples of different types of extracellular targets are provided in Table II, below, along with associated conditions.

TABLE II
Examples of various extracellular targets and associated conditions.

| Target | Disease/Condition |
|---|---|
| TNF receptor | Inflammation, arthritis, autoimmune thyroid |
| | disease, ischemic heart disease |
| TNF-α and β | Inflammation, arthritis, autoimmune thyroid |
| | disease, ischemic heart disease |
| IL-2 receptor | Ischemic heart disease |
| Aldosterone | Cardiovascular heart disease |
| Amyloid beta-peptide | Alzheimer's disease |
| [Abeta(1-42)] | |
| Transthyretin | Alzheimer's disease |
| Erythropoietin | benign erythrocytosis |
| Prostaglandin | Neurodegeneration |
| Cholesterol | Heart disease |
| Retinoid X | hepatogastroenterological diseases |
| Apolipoprotein B-100 | Coronary heart disease |
| Homocysteine | Cardiovascular disease |
| Insulin | Diabetes |
| Apolipoprotein A1 | Heart disease |
| Apolipoprotein CII | Hyperlipidemia |
| Apolipoprotein CII | heart disease |
| Apolipoprotein E | Cardiovascular disease |
| Apolipoprotein E | Alzheimer's disease |
| CD4 | Immune response |
| CD4 receptor | Immune response/HIV infection |
| CCR5 | Immune response/HIV infection |
| SBR1 | HDL receptor/coronary heart disease |
| Annexin V | Clot formation, Apoptosis |
| Fibrin | Wound healing, clot formation |

Among the diffusible extracellular molecules, further subcategories are recognizable. In a preferred embodiment, a target of an engineered polypeptide or polypeptide complex is an extracellular signaling molecule, meaning a molecule that is produced by one cell with the primary effect of triggering a response in another cell. Examples of extracellular signaling molecules include most growth factors and cytokines, neurotransmitters, hormones, and prostaglandins. Many extracellular signaling molecules are actually part of a larger assemblage that carries out the signaling function; for example, TGF-β1 contains two 112 amino acid chains that are linked by a disulfide bond, and either of the two polypeptide chains may be considered to be extracellular signaling molecules that are targeted by an engineered olypeptide or polypeptide complex.

In certain embodiments, an extracellular signaling molecule is a molecule that binds to an extracellular portion of a membrane bound receptor and triggers a signal transduction event in the cell. In certain embodiments, an extracellular signaling molecule is a molecule that enters a cell and binds to an intracellular receptor to trigger a signal transduction event in the cell (e.g., steroid hormones, harpin proteins of various bacterial pathogens).

In a particularly preferred embodiment, the target is an extracellular polypeptide signaling molecule, e.g., as may be found in biological fluid(s), such as a growth factor, cytokine, polypeptide hormone or the like. In certain preferred embodiments, the target is a signaling molecule, particularly a polypeptide signaling molecule, present in serum or other bodily fluid at a concentration of less than 1 μM, and even more preferably less than 0.1 μM, 10 nM, 1 nM, 0.1 nM, 10 pM or even 1 pM. The engineered protein or protein complex may have a catalytic domain chosen so as to modify the signaling molecule in a manner that alters its interaction with a cognate receptor (e.g., abrograting binding or limiting receptor activation), ability to form protein complexes with other soluble factors, half-life and/or biodistribution.

In certain preferred embodiments, the engineered protein or protein complex alters the level of signal transduction induced by an extracellular factor. The term “signal transduction” is intended to encompass the processing of physical or chemical signals from the extracellular environment through the cell membrane and into the cell, and may occur through one or more of several mechanisms, such as activation/inactivation of enzymes (such as proteases, or enzymes which may alter phosphorylation patterns or other post-translational modifications), activation of ion channels or intracellular ion stores, effector enzyme activation via guanine nucleotide binding protein intermediates, second messenger generation (e.g., GTP hydrolysis, calcium mobilization, formation of inositol phosphates, cyclic nucleotides, sugar nucleosides or dissolved gases such as NO or O₃), redistribution of intracellular ions (Ca⁺², Zn⁺², Na⁺, K⁺), and/or direct activation (or inhibition) of a transcriptional factor. Signal transduction may result in physiological changes to the cell, such as changes in morphology, cell adhesion, chemotaxis, drug resistance, growth, proliferation, death (apoptosis or necrosis), effector function, secretion of matrix, etc.

The induction of intracellular signals by the binding of an extracellular signaling molecule, such as a soluble growth factor, to a membrane-spanning receptor is of considerable biological importance. In many cases, promotion of receptor-receptor interactions by protein factors is a key initial step in the induction of a signal transduction process. In certain preferred embodiments, the subject engineered polypeptides or polypeptide complexes can be used to alter the biological function/performance of an inductive protein factor, such as a protein factor selected from one of the protein factor superfamilies known as (i) four-helix bundle factors, (ii) EGF-like factors, (iii) insulin-like factors, (iv) β-trefoil factors and (v) cysteine knot factors. Exemplary targets within in these classes are listed in Table III.

TABLE III
Growth factor structural superfamilies

| Family | Subclass | Examples |
|---|---|---|
| Four-helix bundle | Short chain | IL-2, IL-3, IL-4, IL-5, IL- |
| | | 7, IL-9, IL-13, IL-15, M- |
| | | CSF, GM-CSF |
| | Long chain | GH, LIF, G-CSF, IL-6, IL- |
| | | 12, EPO, OSM, CNTF |
| | Interferon | IFNβ, IFNγ |
| EGF-like | | EGF, TGFα, heregulin |
| Insulin-like | | Insulin, IGF1, IGF2 |
| β-trefoil | | FGF, IL-1 |
| Cysteine knot | | NGF, PDGF, TGFβ |
| | | proteins |

Examples of particular extracellular signaling molecules and conditions associated with these targets which may be treated are listed in Table IV, below.

TABLE IV
Examples of Extracellular Signaling Molecules

| Target | Disease/Condition |
|---|---|
| IL-1α and β | Inflammation, Arthritis, inflammatory bowel |
| | disease |
| IL-4 | Asthma, allergic airway disease |
| IL-5 | Asthma, Allergic airway disease |
| IL-6 | Inflammation, Kaposi's sarcoma |
| IL-7 | Immune response |
| IL-8 | Inflammatory disease, Crohn's disease |
| IL-18 | Arthritis |
| IL-9 | Asthma |
| IL-10 | Colitis |
| IL-11 | Crohn's disease, Ischemic heart disease |
| TNF-α and β | Inflammation, arthritis, autoimmune thyroid |
| | disease, ischemic heart disease |
| VEGF | Cancer, Angiogenesis, Arthritis, Eales' disease |
| Aldosterone | Cardiovascular heart disease |
| Somatostatin | Grave's disease |
| Fibronectin | Ullrich's disease |
| Angiotensin | Heart disease |
| Erythropoietin | Benign erythrocytosis |
| Prostaglandins | Neurodegeneration |
| Interferon α and β | Immune response |
| Retinoid X | hepatogastroenterological diseases |
| Adrenocorticotropic | Cushing's disease |
| Hormone | |
| Hepatocyte growth factor | Cardiovascular disease, periodontal disease |
| Transforming growth | Graft-versus-host disease, renal disease |
| factor-beta1 | |
| Transforming growth | Coeliac Disease |
| factor-beta | |
| Insulin-like growth factor | macrovascular disease and hypertension in |
| binding protein-1 | type 2 diabetes |
| (IGFBP-1) | |
| VEGF-A | Paget's disease |
| Platelet-derived | Paget's disease |
| endothelial cell growth | |
| factor/thymidine | |
| phosphorylase (PD- | |
| ECGF/TP) | |
| Insulin-like growth factor | Inflammatory bowel disease |
| I (IGF-I) | |
| IGF binding protein-3 | Inflammatory bowel disease |
| Insulin | Diabetes |
| EGF | Oncogenesis, Wound healing |
| Vasoactive intestinal | Inflammation |
| peptide | |

In certain particularly preferred embodiments, the target is an inflammatory cytokine, such as tumor necrosis factor (TNF-α), interleukin-6 (IL-6) or interleukin-1b (IL-1b), and the engineered polypeptide or polypeptide complex can be used therapeutically to reduce inflammation.

In certain other preferred embodiments, the target is a polypeptide hormone, such as Adrenocorticotrophic Hormone, Amylin Peptide, Bombesin, Calcitonin, Cholecystokinin (CCK-8), Gastrin, Glicentin, GLP-1, GLP-2, PYY, NPY, GIP, Glucagon, Human Chorionic Gonadotrophin (α), Human Chorionic Gonadotrophin (β), Human Follicle Stimulating Hormone (β2), Human Growth Hormone, Insulin, Luteinising Hormone, Pancreatic Polypeptide, Parathyroid Hormone, Placental Lactogen, Proinsulin, Prolactin, Secretogranin II, Somatostatin, Thyroglobulin, Thyroid Stimulating Hormone, Vasoactive Intestinal Polypeptide.

Other exemplary targets include polypeptide factors selected from the group consisting of: Granulocyte-colony stimulating factor (G-CSF), Myelomonocytic growth factor, Interleukin-3, Interleukin-7, Leukemia inhibitory factor (LIF), Oncostatin M, Ciliary neurotrophic factor (CNTF), cholinergic differentiation factor (CDF), Interleukin-4, Interleukin-13, Interleukin-16, Interleukin-17, Interferon-alpha (IFN-α), Interferon-beta (IFN-β), IFN-tau (IFN-τ), Interferon-omega (IFN-ω), Interleukin-5, Granulocyte-macrophage colony-stimulating factor (GM-CSF), Macrophage colony-stimulating factor (M-CSF), Interleukin-10, Interleukin 1-alpha (IL1-α), Interleukin 1-beta (IL1-β), Gonadotropin, Nerve Growth Factor (NGF), platelet factor 4 (PF-4), bTG, GRO, 9E3, HLA-A2, macrophage inflammatory protein 1 alpha (MIP-1α), macrophage inflammatory protein 1 beta (MIP-1β), Melanoma growth stimulating activity (MGSA), 4-1BB Ligand, ADF, Autocrine Motility Factors, B61, Betacellulin, Cardiotrophin-1, CD27 Ligand, CD30 Ligand, CD40 Ligand, CeK5 Receptor Ligand, EMAP-II, ENA-78, Eosinophil Cationic Protein, Epiregulin, Erythrocyte-derived Growth-Promoting Factor, Erythropoietin, Fas Ligand, Fibrosin, FIC, GDNF, Growth/Differentiation Factor-5, Interleukin-1 Receptor Antagonist, Interleukin-3, Interleukin-6, Interleukin-7, Interleukin-9, Interleukin-11, Interleukin-12, Interleukin-13, Interleukin-14, Interleukin-15, Lymphotactin, LT-beta, Lymphotoxin, MCP-2, MCP-3, Megapoietin, Melanoma-derived Growth Regulatory Protein, Monocyte Chemoattractant Protein-1, Macrophage Migration Inhibitory Factor, Neu Differentiation Factor, Oncostatin M, OX40 Ligand, Placenta Growth Factor, PLF, Scatter Factor, Steel Factor, TCA 3, Thrombopoietin, Vascular Endothelial Cell Growth Factor, Bone Morphogenetic Proteins, Interleukin-1 Receptor Antagonist, Monocyte Chemoattractant Protein-1, c-Kit ligand (stem cell factor), CXC chemokines, CC chemokines, lymphotactin, and C-X3-C chemokines (fractalkine/neurotactin).

In other embodiments, the target is associated with a cell surface, such as a cell surface receptor, ion channel, transporter, adhesion molecule, lipid, or extracellular matrix molecule such as a polysaccharide or glycosaminoglycan.

In certain preferred embodiments, the target is a cell surface receptor protein or ion channel. For instance, the engineered polypeptide or polypeptide complex can be designed to modify a ligand-binding receptor protein in a manner that alters ligand binding kinetics and/or signal transduction activity of the receptor. Receptor proteins which can be substrates include any receptor or channel which interacts with an extracellular molecule (i.e. hormone, growth factor, peptide, ion) to modulate a signal in the cell. To illustrate, the target can be a site on a serpentine receptor (such as G protein coupled receptor), an enzyme-linked receptor (such as a receptor tyrosine kinase, receptor serine/threonine kinase, receptor protein tyrosine phosphatase, receptor guanylyl cyclase, or receptor nitric oxide synthase), or an ion channel (including an ion-channel-linked receptor). Exemplary receptors which can be targeted include cytokine receptors; multisubunit immune recognition receptors (MIRR), chemokine receptors; growth factor receptors, or chemoattracttractant peptide receptors, neuropeptide receptors, light receptors, neurotransmitter receptors, and polypeptide hormone receptors, to name but a few. Further examples of cell surface receptors are provided in Table V, along with associated conditions that may be treated.

TABLE V
Examples of Cell Surface Receptors

| Target | Disease/Condition |
|---|---|
| IL-1 receptor | Inflammation, Arthritis, inflammatory bowel |
| | disease |
| TNE receptor | Inflammation, arthritis, autoimmune thyroid |
| | disease, ischemic heart disease |
| IL-2 receptor | Ischemic heart disease |
| EGF receptor | Cancer |
| Vascular endothelial | Arthritis |
| growth factor receptor | |
| VEGF receptor | Cancer |
| Aldosterone receptor | Cardiovascular heart disease |
| Somatostatin receptor | Grave's disease |
| Fibronectin receptor | Ullrich's disease |
| Angiotensin receptor | Heart disease |
| SBR1 | HDL receptor/coronary heart disease |

Additional examples of cell surface associated or extracellular matrix targets include cellular adhesion molecules, such as selectins, integrins and other hemidesmosomal proteins, cadherins, laminins, CD44 isoforms, proteoglycans (such as syndecans), Ig superfamily (IgCAM) proteins, catenins (such as α, β and γ catenins) and cadherins (such as E-cadherin or P-cadherin), galectins, collagens, elastins, fibrins, and the like.

In certain embodiments, the target is a Cluster of Differentiation (CD) protein, such as CD1a, CD2 (LFA-2), CD3, CD4, CD5, CD6, CD7, CD8, CD9 (Motility-Related Protein-1), CD10 (CALLA), CD11b (Mac-1), CD11b, CD13, CD14, CD15, CD16, CD18 (b2), CD19, CD20, CD21, CD22 (BL-CAM), CD23, CD25 (Interleukin-2 Receptor), CD27, CD29 (b1), CD30, CD31 (PECAM-1), CD34 (Endothelial Cell Marker), CD35, CD37, CD38, CD39, CD40, CD40L (CD154), CD41 (GPIIb/IIIa), CD42b (GPIb), CD43, CD44 (H-CAM), CD44 Variant 3, CD44 Variant 4, CD44 Variant 5, CD44 Variant 6, CD45 (Leucocyte Common Antigen), CD45RA, CD45RB, CD45RO, CD48, CD49b (VLA-2), CD49c (VLA-3), CD49f (VLA-6), CD50 (ICAM-3), CD51, CD54 (ICAM-1), CD56 (NCAM), CD57, CD58 (LFA-3), CD61 (GPIIIa), CD61 (GPIIIa), CD62E (E-selectin), CD62L (L-selectin), CD62P (P-selectin) , CD63 (Melanoma Marker), CD66a (CEACAM1), CD66e (Carcinoembryonic Antigen), CD68, CD69, CD71 (Transferrin Receptor), CD72, CD74, CDw75, CD79a, CD81, CD82, CD83, CD95 (Fas), CD99 (MIC2), CD104, CD105 (Endoglin), CD106 (VCAM-1), CD117 (c-kit Oncoprotein), CD134 (OX40), CD137, CD138 (Syndecan-1), CD141 (Thrombomodulin), CD141 (Thrombomodulin), CD143 (ACE), CD146 (MCAM), CD147 (EMMPRIN), CDw150 (SLAM), CD151 (PETA-3), CD154 (CD40L), CD162, CD163, CD166 (ALCAM), CD168 (RHAMM), or CD179a.

In certain preferred embodiments, the target is a selectin, e.g., a CD62 family protein. In other preferred embodiments, the target is an immunoglobulin superfamily protein (IgCAM), such as a CD2 family protein, CD22, CD31, CD48, CD50, CD54, CD56, CD58, CD66a, CD83, CD106, CD146, CD147, CDw150 or CD166. In still other preferred embodiments, the target is an integrin, such as CD49 family, CD51, CD29, CD11b, CD18, CD41, CD61 or CD104.

Additional targets include the scavenger receptor class A (SR-A, CD204), scavenger receptor-BI (SR-BI) or CD36, which are cell surface proteins that mediate cell adhesion to, and endocytosis of, various native and pathologically modified substances, and participate in intracellular signaling, lipid metabolism, and host defense against bacterial pathogens.

Collagen may also be targeted so as to deep hydrolysis of polypeptide substrates (native or partially denatured collagen types, elastin, fibrin, hemoglobin, and casein). Such engineered polypeptides and complexes have use in both medical and cosmetological applications.

In certain embodiments, the target will be a heteromeric receptor complex, e.g., receptor complexes involving two or more different receptor subunits. For instance, receptors for most interleukins and cytokines that regulate immune and hematopoietic systems belong to the class I cytokine receptor family. These molecules form multichain receptor complexes in order to exhibit high-affinity binding to, and mediate biological functions of, their respective cytokines. In most cases, these functional receptor complexes share common signal transducing receptor components that are also in the class I cytokine receptor family, such as the gp130 protein. Engineered polypeptides or polypeptide complexes which are specifically reactive with the unique receptor subunit(s), but which do not substantially impair the function of the common subunit, can be used to enhance the selectivity as an antagonist of a particular ligand. Alternatively, proteins targeted to selectively inactivate the unique receptor subunits of other ligand-receptor complexes, e.g., those that compete with the formation of receptor complexes for the ligand of interest, can be agonists of ligand of interest.

In still other embodiments, the target is an extracellular molecule that is part of a biomolecular accretion. A biomolecular accretion is any undesirable assemblage of biomolecules, usually one that brings together components that are not typically found in an assemblage together usually one that has grown over time by the successive addition of material. Accretions are generally large enough as to be non-diffusible (although clots are accretions that may diffuse in the circulatory system) and are generally larger than the size of a typical host cell. Biomolecular accretions will often contain dead and living cells as well as extracellular matrix. Examples of biomolecular accretions include amyloid deposits, e.g., a β-amyloid peptide deposit characteristic of Alzheimer's disease or a type II diabetes amyloid deposit, a collagen deposit, a protein deposit, an atherosclerotic plaque, an undesirable fat mass, an undesirable bone mass, a blood clot, or a cyst. Examples of proteins that are often present in the amyloid deposits associated with Alzheimer's disease include amyloid β-peptide [Aβ(1-42)] and transthyretin. Protein aggregation has been linked to several human diseases, including Alzheimer's disease, Parkinson's disease, and systemic amyloidosis. Most of these diseases are associated with the formation of highly ordered and beta-sheet-rich aggregates referred to as amyloid fibrils. Fibril formation by WT transthyretin (TTR) or TTR variants has been linked to systemic amyloidosis and familial amyloid polyneuropathy, respectively. Amyloid fibril formation by α-synuclein (α-syn) has been linked to neurodegeneration in Parkinson's disease. Atherosclerotic plaque may contain a variety of different components. Examples of certain components include: calcified substances (e.g., hydroxyapatite), cholesterol crystals, collagen matrix, macrophage foam cells, smooth muscle cells, lipid-rich atheromatous material (particularly rich in cholesterol and esters thereof), mast cells, matrix metalloproteinases (e.g., MMP-1 collagenase, MMP-2 and -9 gelatinases). Given that atherosclerotic plaque rupture is associated with dangerous thrombotic events, it may be desirable to design an protein or protein complex that stabilizes plaques (e.g. by targeting metalloproteinases in the plaque) or to employ a plaque-dissolving protein in combination with an anti-thrombotic agent, such as heparin.

(b) Intracellular Targets

In certain embodiments, a target may be an intracellular target. Examples of intracellular targets include intracellular receptors (e.g., many steroid hormone receptors), enzymes that are overexpressed or otherwise participate in an undesirable condition, intracellular signaling proteins that participate in an undesirable condition (e.g., oncoproteins, pro-inflammatory proteins) and transcription factors.

In an exemplary embodiment, the target is a nuclear receptor. Many nuclear receptors may be viewed as ligand-dependent transcription factors. These receptors provide a direct link between extracellular signals, mainly hormones, and transcriptional responses. Their transcriptional activation function is regulated by endogenous small molecules, such as steroid hormones, vitamin D, ecdysone, retinoic acids and thyroid hormones, which pass readily through the plasma membrane and bind their receptors inside the cell. The subject polypeptides and polypeptide complexes can be used, for example, to alter the responsiveness of a cell to a particular hormone or other nuclear receptor ligand, such as by degrading receptor complexes to inhibit response to a hormone of interest, or degrading subunits for other receptor dimers that otherwise compete with the formation of receptor complexes for the hormone of interest.

Examples of certain intracellular targets are provided in Table VI, along with associated conditions that may be treated.

TABLE VI
Examples of Intracellular Targets

| Target | Disease/Condition |
|---|---|
| aldosterone receptor | Cardiovascular heart disease |
| Erythropoietin | benign erythrocytosis |
| PPAR γ | hepatogastroenterological diseases |
| Adrenocorticotropic | Cushing's disease |
| Hormone | |
| Huntingtin protein | Huntington's disease |
| estrogen receptor | Coronary heart disease, Liver disease |
| glucose-6-phosphatase | Glycogen storage disease type 1 |
| erythrocyte antioxidant | Behcet's disease |
| enzyme | |
| androgen receptor | Paget's disease |
| platelet-derived | Paget's disease |
| endothelial cell growth | |
| factor/thymidine | |
| phosphorylase (PD- | |
| ECGF/TP) | |
| Rb | Cancer |
| P16 | Cancer |
| P21 | Cancer |
| P53 | Cancer |
| HIF-1 | Cancer |
| NF-κB | Inflammatory disease |
| NF-κB | Cell Death |
| IκB | Immune response |

In embodiments involving an intracellular target, it will generally be desirable to further engineer a polypeptide or polypeptide complex such that it is produced within cells or designed for entry into cells. This may be accomplished by including one or more functionalities that promote uptake by target cells, e.g., promote the initial step of uptake from the extracellular environment. In one embodiment, a subject engineered polypeptide or polypeptide complex includes an “internalizing peptide” which drives the translocation of the engineered polypeptide or polypeptide complex across a cell membrane in order to facilitate intracellular localization. The internalizing peptide, by itself, is capable of crossing a cellular membrane by, e.g., transcytosis, at a relatively high rate. In certain embodiments, engineered polypeptides may be expressed from a nucleic acid that is introduced into a cell, such as a viral vector or naked or encapsulated nucleic acid vector.

In one embodiment, an internalizing peptide is derived from the Drosophila antepennepedia protein, or homologs thereof. The 60 amino acid long homeodomain of the homeo-protein antepennepedia has been demonstrated to translocate through biological membranes and can facilitate the translocation of heterologous peptides and organic compounds to which it is couples. See for example Derossi et al. (1994) J Biol Chem 269:10444-10450; and Perez et al. (1992) J Cell Sci 102:717-722. Recently, it has been demonstrated that fragments as small as 16 amino acids long of this protein are sufficient to drive internalization. See Derossi et al. (1996) J Biol Chem 271:18188-18193.

Another example of an internalizing peptide is the HIV transactivator (TAT) protein. This protein appears to be divided into four domains (Kuppuswamy et al. (1989) Nucl. Acids Res. 17:3551-3561). Purified TAT protein is taken up by cells in tissue culture (Frankel and Pabo, (1989) Cell 55:1189-1193), and peptides, such as the fragment corresponding to residues 37-62 of TAT, are rapidly taken up by cell in vitro (Green and Loewenstein, (1989) Cell 55:1179-1188). The highly basic region mediates internalization and targeting of the internalizing moiety to the nucleus (Ruben et al., (1989) J. Virol. 63:1-8).

A sufficient portion of mastoparan (T. Higashijima et al., (1990) J. Biol. Chem. 265:14176) may be used to increase the transmembrane transport of the polypeptide or complex.

(c) Infective or Foreign Targets

An additional category of targets includes targets that are associated with an infective or otherwise undesirable foreign agent, such as protists, yeasts, bacteria, viruses and prions and various complexes. In certain embodiments, an engineered polypeptide or polypeptide complex is targeted against a virulence factor that is exposed on the surface of a bacterium, such as a pilin or other adhesive protein, a flagellin, or other motility protein, a protein that facilitates bacterial cell entry into the host cell cytoplasm. In certain embodiments, the engineered polypeptide or polypeptide complex may disrupt a structural component of a bacterial cell wall or membrane, sufficient to cause cell lysis. In certain embodiments, the target may be a protein or other component of a virus that is required for viral particle viability or entry into a host cell, e.g., a protein of a viral coat or envelope. In another example, the target may be a toxin, a venom, an undesirable foreign chemical or a heavy metal.

(d) Molecules Targeted by Developed Therapeutic Agents

One novel approach to selecting an appropriate recipient and target is to identify molecules that are targeted by therapeutically active agents that act by binding to the targeted molecules, such as monoclonal antibodies and soluble receptor portions. In a preferred embodiment, the target molecule is a target for a FDA-approved, commercially available therapeutic binding agent. It is expected that a binding agent may be improved or modified by the addition of an engineered motif, such as a catalytic motif that is active against the target.

In certain embodiments, the target is CD52, which is involved in B cell chronic lymphocytic lymphoma (CLL). CD52 is a 21-28 kD glycoprotein expressed on the surface of normal and malignant B and T lymphocytes, NK cells, monocytes, macrophages, and tissues of the male reproductive system. Campath® (Alemtuzumab) is a recombinant DNA-derived humanized CD52 monoclonal antibody (Campath-1H). One problem associated with the use of Campath is hematologic toxicity, which tend to occur when single doses of greater than 30 mg or cumulative doses greater than 90 mg per week are administered. Thus an engineered variant with catalytic activity may be administered at a lower dose because of its catalytic nature, providing a better therapeutic alternative. The recipient may be the same monoclonal antibody or functional derivative thereof (such as a scFv derivative as in the instant application).

In certain embodiments, the engineered polypeptide or polypeptide complex is an antagonist of TNF-alpha. Such engineered polypeptides or polypeptide complexes can be used as part of a treatment for Rheumatoid arthritis, inflammatory bowel disease (IBD), including Crohn's disease and and ulcerative colitis. Human TNF-alpha is a non-glycosylated protein of 17 kDa, while murine TNF-alpha is N-glycosylated. TNF-alpha shows a wide spectrum of biological activities, and is found to be the important part of the whole IBD problem. Enbrel (etanercept; Immunex) and Remicade (infliximab; Centocor) are TNF-alpha antibodies that are used for severe cases of Rheumatoid arthritis and Crohn disease. The two drugs are very similar in mechanism, as is Humira (adalimumab; Abbott), a very recently approved TNF antibody which is much more faithful to human antibody structure. A subject engineered polypeptide or polypeptide complex, which can be administered at a much lower dose because of its catalytic nature, is expected to be a better therapeutic alternative. The recipient may be the same monoclonal antibody or functional derivative thereof (such as a scFv derivative as in the instant application).

In certain embodiments, the engineered polypeptide or polypeptide complex is an antagonist of the HER2/neu receptor. Such engineered polypeptides or polypeptide complexes can be used as part of a treatment for metastatic breast cancer and/or recurrent or refractory ovarian or primary peritoneal carcinoma with overexpression of HER2. The HER2 (or c-erbB2) proto-oncogene encodes a transmembrane receptor protein of 185 kDa, which is structurally related to the epidermal growth factor receptor 1 (EGFR1). HER2 protein overexpression is observed in 25%-30% of primary breast cancers. HERCEPTIN (Trastuzumab) is a recombinant DNA-derived humanized monoclonal antibody that selectively binds with high affinity in a cell-based assay (K_{d}=5 nM) to the extracellular domain of HER2. The antibody is a humanized murine IgG1 kappa. One problem associated with the use of HERCEPTIN administration is severe hypersensitivity reactions (including anaphylaxis), infusion reactions, and pulmonary events. Thus a subject engineered polypeptide or polypeptide complex, which can be administered at a much lower dose because of its catalytic nature, is expected to be a better therapeutic alternative. The recipient may be the same monoclonal antibody or functional derivative thereof (such as a scFv derivative as in the instant application).

In certain embodiments, the engineered polypeptide or polypeptide complex is an antagonist of CD33. Such engineered polypeptides or polypeptide complexes can be used as part of a treatment for Acute myeloid leukemia (AML), the most common type of acute leukemia in adults. CD33 antigen is a sialic acid-dependent adhesion protein found on the surface of leukemic blasts and immature normal cells of myelomonocytic lineage, but not on normal hematopoietic stem cells. “Mylotarg” (gemtuzumab ozogamicin for Injection) is a chemotherapy agent composed of a recombinant humanized IgG4, kappa antibody conjugated with a cytotoxic antitumor antibiotic, calicheamicin, isolated from fermentation of a bacterium, Micromonospora echinospora ssp. calichensis. The antibody portion of Mylotarg binds specifically to the CD33 antigen. Side effects associated with the use of Mylotarg includes hypersensitivity reactions, including anaphylaxis, infusion reactions, pulmonary events, and hepatotoxicity. Thus a subject engineered polypeptide or polypeptide complex, which can be administered at a much lower dose because of its catalytic nature, is expected to be a better therapeutic alternative. The recipient may be the same monoclonal antibody or functional derivative thereof (such as a scFv derivative as in the instant application).

In certain embodiments, the engineered polypeptide or polypeptide complex is an antagonist of CD3. Such engineered polypeptides or polypeptide complexes can be used as part of a treatment for transplant rejection, such as acute renal, steroid-resistant cardiac, or steroid-resistant hepatic allograft rejection. OKT3 (or “muromonab-CD3”) is a murine monoclonal antibody to the CD3 antigen of human T cells which functions as an immunosuppressant. The antibody is a biochemically purified IgG2a immunoglobulin. It is directed to the CD3 glycoprotein in the human T cell surface which is essential for T cell functions. Modulated cells, which reversibly lose the expression of the CD3 T cell receptor molecular complex but still share the CD4 and CD8 antigens, have been shown to be functionally immunoincompetent. Thus the subject engineered polypeptide or polypeptide complex, which can be administered at a much lower dose because of its catalytic nature, is expected to be a better therapeutic alternative. The recipient may be the same monoclonal antibody or functional derivative thereof (such as a scFv derivative as in the instant application).

In certain embodiments, the engineered polypeptide or polypeptide complex is an antagonist of gpIIb/IIIa. Such engineered polypeptides or polypeptide complexes can be used as part of a treatment for Acute myocardial infarction/unstable angina. Abciximab (ReoPro®), is the Fab fragment of the chimeric human-murine monoclonal antibody 7E3. Abciximab binds to the glycoprotein (GP) IIb/IIIa (a_{IIb}b₃) receptor of human platelets and inhibits platelet aggregation. Abciximab also binds to the vitronectin (aᵥb₃) receptor found on platelets and vessel wall endothelial and smooth muscle cells. A subject engineered polypeptide or polypeptide complex, which can be administered at a much lower dose because of its catalytic nature, is expected to be a better therapeutic alternative. The recipient may be the same monoclonal antibody or functional derivative thereof (such as a Fab or scFv derivative as in the instant application).

In certain embodiments, the engineered polypeptide or polypeptide complex is an antagonist of CD20. Such engineered polypeptides or polypeptide complexes can be used as part of a treatment for Non-Hodgkin's lymphoma (NHL), such as CD20 positive, follicular, Non-Hodgkin's lymphoma. The CD20 antigen is found on the surface of normal and malignant B lymphocytes. The RITUXAN® (Rituximab) antibody is a genetically engineered chimeric murine/human monoclonal antibody directed against the CD20 antigen found on the surface of normal and malignant B lymphocytes. The antibody is an IgG1 kappa immunoglobulin containing murine light- and heavy-chain variable region sequences and human constant region sequences. Rituximab has a binding affinity for the CD20 antigen of approximately 8.0 nM. A second approved drug, ZEVALIN (Ibritumomab Tiuxetan), is the immunoconjugate resulting from a stable thiourea covalent bond between the monoclonal antibody Ibritumomab and the linker-chelator tiuxetan [N-[2-bis(carboxymethyl)amino]-3-(p-isothiocyanatophenyl)-propyl]-[N-[2-bis(carboxymethyl)amino]-2-(methyl)-ethyl]glycine. This linker-chelator provides a high affinity, conformationally restricted chelation site for Indium-111 or Yttrium-90. The antibody moiety of ZEVALIN is Ibritumomab, a murine IgG1 kappa monoclonal antibody directed against the CD20 antigen. A third drug, Bexxar (tositumomab and iodine-131 tositumomab), is another approved drug for the treatment of patients with CD20 positive, follicular, Non-Hodgkin's lymphoma, with and without transformation, whose disease is refractory to Rituxan and has relapsed following chemotherapy. The subject engineered polypeptide or polypeptide complex, which can be administered at a much lower dose because of its catalytic nature, is expected to be a better therapeutic alternative. The recipient may be the same monoclonal antibodies or functional derivative thereof (such as a Fab or scFv derivative as in the instant application).

In certain embodiments, the engineered polypeptide or polypeptide complex is an antagonist of RSV F Protein. Such engineered polypeptides or polypeptide complexes can be used as part of a treatment for RSV infection. SYNAGIS® (PALIVIZUMAB) is a humanized monoclonal antibody (IgG1k) produced by recombinant DNA technology, directed to an epitope in the A antigenic site of the F protein of respiratory syncytial virus (RSV). Palivizumab is a composite of human (95%) and murine (5%) antibody sequences. A subject engineered polypeptide or polypeptide complex, which can be administered at a much lower dose because of its catalytic nature, is expected to be a better therapeutic alternative. The recipient may be the same monoclonal antibody or functional derivative thereof (such as a Fab or scFv derivative as in the instant application).

In certain embodiments, the engineered polypeptide or polypeptide complex is an antagonist of CD25. Such engineered polypeptides or polypeptide complexes can be used as part of a treatment for transplant rejection. Zenapax® (daclizumab) is an immunosuppressive, humanized IgG1 monoclonal antibody produced by recombinant DNA technology that binds specifically to the alpha subunit )p55 alpha, CD25, or Tac subunit) of the human high affinity IL-2 receptor that is expressed on the surface of activated (but not resting) lymphocytes. The drug binds to the high affinity IL-2 receptor, thus inhibiting the binding of Tac by IL-2, and the activation of lymphocytes. Therefore, the monoclonal antibody acts as a pure binder inhibitor. The subject engineered polypeptide or polypeptide complex, which can be administered at a much lower dose because of its catalytic nature, is expected to be a better therapeutic alternative. The recipient may be the same monoclonal antibody or functional derivative thereof (such as a Fab or scFv derivative as in the instant application).

In certain embodiments, the engineered polypeptide or polypeptide complex is an antagonist of IL-1. Such engineered polypeptides or polypeptide complexes can be used as part of a treatment for Rheumatoid arthritis. The pathogenesis of RA is a complex process that leads to significant and chronic joint inflammation. Interleukin-1 (IL-1) is a central mediator in RA and is a critical proinflammatory cytokine that has been found to be abundant in the synovial fluid of RA patients. Kineret® (anakinra) is a recombinant, nonglycosylated form of the human interleukin 1 receptor antagonist (IL-1Ra). Kineret® differs from native human IL-1Ra in that it has the addition of a single methionine residue at its amino terminus. Kineret® blocks the biologic activity of IL-1 by competitively inhibiting IL-1 binding to the interleukin-1 type I receptor (IL-1RI), which is expressed in a wide variety of tissues and organs. Therefore, Kineret® acts as a pure binder inhibitor. The subject engineered polypeptide or polypeptide complex, which can be administered at a much lower dose because of its catalytic nature, is expected to be a better therapeutic alternative. The recipient may be the same monoclonal antibody or functional derivative thereof (such as a Fab or scFv derivative as in the instant application). A panel of proteases that are capable of efficiently digesting IL-1 may be used as the catalytic domain.

In certain embodiments, IgE (immunoglobulin E) may be the target of an engineered polypeptide or polypeptide complex. IgE is a class of antibodies that protects the host against invading parasites. IgE interacts with mast cells and eosinophils to protect the host against the invading parasite. The IgE-immune cell complex is also responsible for many allergic or hypersensitivity reactions such as hay fever, asthma, hives and anaphylaxis. There are two major types of receptor for the Fc portion of the IgE on cells. A high affinity receptor is found primarily on mast cells and basophils. A low affinity receptor is found on CD23 cells. IgE attaches to these and acts as an antigen receptor. Xolair™ is a humanized monoclonal antibody directed to the Fc portion of IgE and effective in treating asthma. An engineered polypeptide or polypeptide complex targeted to and reducing the activity of IgEs (generally by targeting the Fc portion) may be used to treat asthma. The recipient may be a monoclonal antibody or functional derivative thereof (such as a scFv derivative as in the instant application), or a soluble ligand binding portion of an IgE receptor.

In certain embodiments, VEGF (Vascular Endothelial Growth Factor) may be the target of an engineered polypeptide or polypeptide complex. VEGF plays a critical role in angiogenesis (the formation of new blood vessels), particularly in tumors and is also involved in the maintenance of established tumor blood vessels. VEGF is homodimeric and disulfide linked. Four human splice variants of VEGF have been identified encoding, in the mature form, polypeptide monomers of 121, 165, 189, or 206 amino acids. Two receptor tyrosine kinases (RTKs), Flt-1 and Flk-1 bind VEGF with high affinity. Avastin™ is an investigational recombinant humanized antibody to VEGF, and shows effectiveness in improving the survival of metastatic colorectal cancer patients. An engineered polypeptide or polypeptide complex targeted to and reducing the activity of VEGF may be used to treat a variety of cancers, particularly colorectal cancer. The recipient may be a monoclonal antibody or functional derivative thereof (such as a scFv derivative as in the instant application), or a soluble ligand binding portion of a VEGF receptor.

In certain embodiments, EGFR (Epidermal Growth Factor Receptor) may be the target for an engineered polypeptide or polypeptide complex. EGFR is expressed in a high percentage of many cancer types, including head and neck, colorectal, pancreatic, lung, esophageal, renal cell, prostate, bladder, cervical/uterus, ovarian and breast cancers. ERBITUX™ (formerly known as IMC-C225) is a highly specific chimerized monoclonal antibody that binds to EGFR and blocks the ability of EGF to initiate receptor activation and signaling to the tumor. This blockade results in an inhibition of tumor growth by interfering with the effects of EGFR activation including tumor invasion and metastases, cell repair and angiogenesis. ERBITUX™ has been used in combination with chemotherapy and radiation in animal models of human cancers. These preclinical findings indicate that when combined with chemotherapy or radiation, ERBITUX™ treatment provides an enhanced anti-tumor effect resulting in the elimination of tumors and the long-term survival of the animals. An engineered polypeptide or polypeptide complex targeted to and reducing the presence, ligand-binding or signaling capacity of EGFR may be used to treat or prevent a variety of cancers, particularly colorectal cancer, and particularly when used in combination with one or more additional chemotherapeutic agents. The recipient may be a monoclonal antibody or functional derivative thereof (such as a scFv derivative as in the instant application), or a soluble ligand (such as EGF) for EGFR.

In certain embodiments, one or more alpha-4 integrins, such as beta-1 and beta-7 may be the target(s) for an engineered polypeptide or polypeptide complex. Integrins are transmembrane proteins, and the alpha-4-beta 1 (VLA-4) and alpha-4-beta-7 integrins help white blood cells, particularly T lymphocytes and eosinophils, move from through the blood vessel walls into the tissues of the body at sites of inflammation, where these cells then participate in the inflammatory process. Antegren® is a humanized monoclonal antibody that binds to and blocks both the beta-1 and beta-7 integrins, preventing the contribution of many cell types to inflammation; Antegren® shows effectiveness for treatment of Crohn's disease. An engineered polypeptide or polypeptide complex targeted to and reducing the presence or ligand-binding capacity of these integrins may be used to treat or prevent a variety of inflammatory diseases, particularly Crohn's disease. The recipient may be a monoclonal antibody or functional derivative thereof (such as a scFv derivative as in the instant application), or a soluble ligand for the targeted alpha-4 integrins.

In certain embodiments, CCR-5 may be the target of an engineered polypeptide or polypeptide complex. The human CCR5 chemokine receptor is a member of the rhodopsin superfamily of G-linked receptors having seven hydrophobic transmembrane domains. CCR5 binds RANTES, MIP-1β and MIP-1α. Raport, C. J. et al. (1996) J. Biol. Chem. 271:17161. CCR5 facilitates infection by the macrophage-tropic HIV-1 virus, RANTES, MIP-1α and MIP-1β can suppress the infection of susceptible cells by macrophage-tropic HIV-1 isolates. Choe, H. et al. (1996) Cell 85:1135. Cocchi, F. et al. (1995) Science 270:1811. Although no CCR-5 targeted affinity agent has been approved, CCR-5 is implicated in HIV infection, and an engineered polypeptide or polypeptide complex targeted to and reducing the presence or HIV-binding capacity of CCR-5 may be used to treat or prevent asthma and other allergic reactions. The recipient may be a monoclonal antibody or functional derivative thereof (such as a scFv derivative as in the instant application), or a soluble ligand for CCR-5.

In certain embodiments, interleukin-4 may be the target of an engineered polypeptide or polypeptide complex. Human IL-4 is a pleiotropic cytokine produced by activated T cells, mast cells, and basophils. The biological effects of IL-4 are mediated by the binding of IL-4 to specific cell surface receptors. The functional high-affinity receptor for IL-4 includes a ligand binding subunit (IL-4 R) and a second subunit (β chain) that can modulate the ligand binding affinity of the receptor complex. The gamma chain of the IL-2 receptor complex may also be a functional β chain of the IL-4 receptor complex. Mature IL-4 is a 129 amino acid protein Yokota, T. et al., 1986, Proc. Natl. Acad. Sci. USA 83:5894. IL-4 activity may be measured, for example, in a cell proliferation assay employing a human factor-dependent cell line, TF-1. Kitamura et al., 1989 J. Cell Physiol. 140:323. Although no IL-4 targeted affinity agent has been approved, IL-4 is implicated in allergies and asthma, and an engineered polypeptide or polypeptide complex targeted to and reducing the activity of IL-4 may be used to treat or prevent asthma and other allergic reactions. The recipient may be a monoclonal antibody or functional derivative thereof (such as a scFv derivative as in the instant application), or a soluble ligand binding portion of an IL-4 receptor.

In certain embodiments, IL-13 may be the target of an engineered polypeptide or polypeptide complex. Although no IL-13 targeted affinity agent has been approved, IL-13 is widely recognized as a cytokine that is involved in asthma and various allergies. Mature human IL-13 is a 112 amino acid polypeptide having a sequence as described in GenBank accession no. P35225. McKenzie et al. 1993, PNAS USA 90:3735-3739. IL-13 activity may be measured, for example, in a cell proliferation assay employing a human factor-dependent cell line, TF-1. Kitamura et al., 1989 J. Cell Physiol. 140:323. An engineered polypeptide or polypeptide complex targeted to and reducing the activity of IL-13 may be used to treat or prevent asthma and other allergic reactions. The recipient may be a monoclonal antibody or functional derivative thereof (such as a scFv derivative as in the instant application), or a soluble ligand binding portion of an IL-13 receptor.

(i) TNFα Antagonists

In certain embodiments, the subject engineered polypeptide or polypeptide complex is a TNFα antagonist. TNFα is a soluble homotrimer of 17 kD protein subunits. A membrane-bound 26 kD precursor form of TNFα also exists. The pleiotropic activities of the potent proinflammatory cytokine TNF are mediated by two structurally related, but functionally distinct, receptors, p55 and p75, that are coexpressed on most cell types. To exert its biological activity, TNFα (a homotrimeric molecule) must bind to at least 2 cell surface receptors, causing cross-linking and cell signaling. The majority of biologic responses classically attributed to TNFα are mediated by p55. In contrast, p75 has been proposed to function as both a TNF antagonist by neutralizing TNFα and as a TNFα agonist by facilitating the interaction between TNFα and p55 at the cell surface. The roles of p55 and p75 in mediating and modulating the activity of TNFα in vivo have been examined in mice genetically deficient in these receptors. Selective deficits in several host defense and inflammatory responses are observed in mice lacking p55 or both p55 and p75, but not in mice lacking p75. In these models, the activity of p55 is not impaired by the absence of p75, arguing against a physiologic role for p75 as an essential element of p55-mediated signaling. In contrast, exacerbated pulmonary inflammation and dramatically increased endotoxin induced serum TNFα levels in mice lacking p75 suggest a dominant role for p75 in suppressing TNFα-mediated inflammatory responses.

The p55 receptor (also termed TNF-R55, TNF-RI, or TNFRα) is a 55 kd glycoprotein shown to transduce signals resulting in cytotoxic, antiviral, and proliferative activities of TNFα. The p75 receptor (also termed TNF-R75, TNF-RII, or TNFRα) is a 75 kDa glycoprotein that has also been shown to transduce cytotoxic and proliferative signals as well as signals resulting in the secretion of GM-CSF. The extracellular domains of the two receptors have 28% homology and have in common a set of four subdomains defined by numerous conserved cysteine residues. The p75 receptor differs, however, by having a region adjacent to the transmembrane domain that is rich in proline residues and contains sites for O-linked glycosylation. Interestingly, the cytoplasmic domains of the two receptors share no apparent homology which is consistent with observations that they can transduce different signals to the interior of the cell.

In certain preferred embodiments, a recipient for generating an engineered polypeptide or complex that targets TNF-α may be derived from the extracellular ligand binding domain of the p75 or p55 receptor, e.g., a portion sufficient to specifically bind to TNF-α. For instance, the recipient can include a ligand binding fragment of p75, such as from Leu23-Asp257 of the human p75 protein (Swiss-Prot Accession P20333) or a ligand binding fragment of p55, such as from Ile22-Thr211 of the human p55 protein (Swiss-Prot Accession P19438). In certain embodiments, the subject engineered polypeptides or polypeptide complexes can be generated from Onercept (a fully human soluble fragment of p55) or Etanercept (Enbrele, a dimeric construct in which two p75 extracellular fragments are linked to the Fc portion of human IgG1).

In other preferred embodiments, the recipient is derived from an antibody that binds to TNFα, or an antigen binding domain thereof. For instance, the subject engineered polypeptides or polypeptide complexes can generated using the monoclonal anti-TNFα antibody is infliximab (Remicade®), or the variable domains of one or both of the heavy and light chains thereof, such as the Fv fragment. Infliximab is a chimeric human/mouse monoclonal anti-TNFα antibody composed of the constant regions of human (Hu) IgG1κ, coupled to the Fv region of a high-affinity neutralizing murine anti-HuTNFa antibody. Likewise, the subject engineered polypeptide or polypeptide complex can be derived from the human anti-TNF antibody D2E7, also known as adalumimab.

The ability of any particular engineered polypeptide or polypeptide complex to act alter the activity of TNFα can be assayed using any of a variety of cell-based and cell-free assay systems well known in the art. Exemplary assays include, but are not limited to, L929 assay, endothelial procoagulation assays, tumor fibrin deposition assays, cytotoxicity assay, tumor regression assays, receptor binding assays, arthritic index assays in mouse model systems, and the like. In certain preferred embodiments of TNFα antagonist engineered polypeptides or polypeptide complexes, their biological activities will include one or more of: inhibition of TNF-α cytotoxicity in L929 cells; blocking of prostaglandin E2 production and expression of cell-associated IL1 by human dermal fibroblasts; blocking of TNF-α binding to the promonocytic cell line U937; blocking of TNF-α induced respiratory burst in human neutrophils; blocking of TNF-stimulated neutrophil lucigenin-dependent chemiluminescence response and superoxide formation; significantly reducing the priming ability of TNF-α for a response to the chemotactic peptide fMLP; blocking of class I antigen expression in the human Colo 205 tumor cell line; affecting TNF-α synergism with HLA-DR antigen expression induced by IFN-γ (yet preferably having no effect on IFN-γ activity).

The subject TNFα antagonist engineered polypeptides or polypeptide complexes can be used to treat various TNF-associated disorders, e.g., disorders or diseases that are associated with, result from, and/or occur in response to, elevated levels of TNFα. Such disorders may be associated with episodic or chronic elevated levels of TNFα activity and/or with local or systemic increases in TNFα activity. Such disorders include, but are not limited to, inflammatory diseases, such as arthritis and inflammatory bowel disease, and congestive heart failure.

TNFα causes pro-inflammatory actions which result in tissue injury, such as degradation of cartilage and bone, induction of adhesion molecules, inducing procoagulant activity on vascular endothelial cells, increasing the adherence of neutrophils and lymphocytes, and stimulating the release of platelet activating factor from macrophages, neutrophils and vascular endothelial cells. In certain preferred embodiments, the TNFα antagonist engineered polypeptide or polypeptide complex reduces the inflammatory activity of TNFα.

Recent evidence also associates TNFα with infections, immune disorders, neoplastic pathologies, autoimmune pathologies and graft-versus-host pathologies. For instance, TNFα is understood to play a central role in gram-negative sepsis and endotoxic shock, including fever, malaise, anorexia, and cachexia. Endotoxin strongly activates monocyte/macrophage production and secretion of TNFα and other cytokines (Kombluth et al., J. Immunol. 137:2585-2591 (1986)). Circulating TNFα levels increase in patients suffering from gram-negative sepsis. Thus, the subject TNFα antagonist engineered polypeptides or polypeptide complexes may used as part of a treatment protocol for inflammatory diseases, autoimmune diseases, viral, bacterial and parasitic infections, malignancies, and neurogenerative diseases, such as for therapy in rheumatoid arthritis and Crohn's disease.

There is evidence that TNFα is also involved in cachexia in cancer, infectious pathology, and other catabolic states. Accordingly, the TNFα antagonist engineered polypeptides or polypeptide complexes can also be used to reduce muscle wasting associated with such disorders, or any other in which cachexia is an issue in patient management.

Accordingly, the present invention provides methods in which the subject engineered polypeptides or polypeptide complexes can be used as part of treatments for modulating or reducing the severity of at least one immune related disease, in a cell, tissue, organ, animal, or patient including, but not limited to, at least one of rheumatoid arthritis, juvenile rheumatoid arthritis, systemic onset juvenile rheumatoid arthritis, psoriatic arthritis, ankylosing spondilitis, gastric ulcer, seronegative arthropathies, osteoarthritis, inflammatory bowel disease, ulcerative colitis, systemic lupus erythematosis, antiphospholipid syndrome, iridocyclitis/uveitis/optic neuritis, idiopathic pulmonary fibrosis, systemic vasculitis/wegener's granulomatosis, sarcoidosis, orchitis/vasectomy reversal procedures, allergic/atopic diseases, asthma, allergic rhinitis, eczema, allergic contact dermatitis, allergic conjunctivitis, hypersensitivity pneumonitis, transplants, organ transplant rejection, graft-versus-host disease, systemic inflammatory response syndrome, sepsis syndrome, gram positive sepsis, gram negative sepsis, culture negative sepsis, fungal sepsis, neutropenic fever, urosepsis, meningococcemia, trauma/hemorrhage, burns, ionizing radiation exposure, acute pancreatitis, adult respiratory distress syndrome, rheumatoid arthritis, alcohol-induced hepatitis, chronic inflammatory pathologies, sarcoidosis, Crohn's pathology, sickle cell anemia, diabetes, nephrosis, atopic diseases, hypersensitity reactions, allergic rhinitis, hay fever, perennial rhinitis, conjunctivitis, endometriosis, asthma, urticaria, systemic anaphalaxis, dermatitis, pernicious anemia, hemolytic disesease, thrombocytopenia, graft rejection of any organ or tissue, kidney translplant rejection, heart transplant rejection, liver transplant rejection, pancreas transplant rejection, lung transplant rejection, bone marrow transplant (BMT) rejection, skin allograft rejection, cartilage transplant rejection, bone graft rejection, small bowel transplant rejection, fetal thymus implant rejection, parathyroid transplant rejection, xenograft rejection of any organ or tissue, allograft rejection, anti-receptor hypersensitivity reactions, Graves disease, Raynoud's disease, type B insulin-resistant diabetes, asthma, myasthenia gravis, antibody-meditated cytotoxicity, type III hypersensitivity reactions, systemic lupus erythematosus, POEMS syndrome (polyneuropathy, organomegaly, endocrinopathy, monoclonal gammopathy, and skin changes syndrome), polyneuropathy, organomegaly, endocrinopathy, monoclonal gammopathy, skin changes syndrome, antiphospholipid syndrome, pemphigus, scleroderma, mixed connective tissue disease, idiopathic Addison's disease, diabetes mellitus, chronic active hepatitis, primary billiary cirrhosis, vitiligo, vasculitis, post-MI cardiotomy syndrome, type IV hypersensitivity, contact dermatitis, hypersensitivity pneumonitis, allograft rejection, granulomas due to intracellular organisms, drug sensitivity, metabolic/idiopathic, Wilson's disease, hemachromatosis, alpha-1-antitrypsin deficiency, diabetic retinopathy; Hashimoto's thyroiditis, osteoporosis, hypothalamic-pituitary-adrenal axis evaluation, primary biliary cirrhosis, thyroiditis, encephalomyelitis, cachexia, cystic fibrosis, neonatal chronic lung disease, chronic obstructive pulmonary disease (COPD), familial hematophagocytic lymphohistiocytosis, dermatologic conditions, psoriasis, alopecia, nephrotic syndrome, nephritis, glomerular nephritis, acute renal failure, hemodialysis, uremia, toxicity, preeclampsia, okt3 therapy, anti-cd3 therapy, cytokine therapy, chemotherapy, radiation therapy (e.g., including but not limited to asthenia, anemia, cachexia, and the like), chronic salicylate intoxication, and the like.

In one embodiment, a TNFα engineered polypeptide or polypeptide complex is used to treat hypergastrinemia, such as Helicobacter Pylori-induced gastritis.

The present invention also provides methods for using the subject TNFα antagonist engineered polypeptides or polypeptide complexes for modulating or treating at least one cardiovascular disease in a cell, tissue, organ, animal, or patient, including, but not limited to, at least one of cardiac stun syndrome, myocardial infarction, congestive heart failure, stroke, ischemic stroke, hemorrhage, arteriosclerosis, atherosclerosis, restenosis, diabetic ateriosclerotic disease, hypertension, arterial hypertension, renovascular hypertension, syncope, shock, syphilis of the cardiovascular system, heart failure, cor pulmonale, primary pulmonary hypertension, cardiac arrhythmias, atrial ectopic beats, atrial flutter, atrial fibrillation (sustained or paroxysmal), post perfusion syndrome, cardiopulmonary bypass inflarnmation response, chaotic or multifocal atrial tachycardia, regular narrow QRS tachycardia, specific arrythmias, ventricular fibrillation, His bundle arrhythmias, atrioventricular block, bundle branch block, myocardial ischemic disorders, coronary artery disease, angina pectoris, myocardial infarction, cardiomyopathy, dilated congestive cardiomyopathy, restrictive cardiomyopathy, valvular heart diseases, endocarditis, pericardial disease, cardiac tumors, aordic and peripheral aneuryisms, aortic dissection, inflammation of the aorta, occulsion of the abdominal aorta and its branches, peripheral vascular disorders, occulsive arterial disorders, peripheral atherlosclerotic disease, thromboangitis obliterans, functional peripheral arterial disorders, Raynaud's phenomenon and disease, acrocyanosis, erythromelalgia, venous diseases, venous thrombosis, varicose veins, arteriovenous fistula, lymphedema, lipedema, unstable angina, reperfusion injury, post pump syndrome, ischemia-reperfusion injury, and the like.

The present invention also provides methods using the subject TNFα antagonist engineered polypeptides or polypeptide complexes for modulating or treating at least one infectious disease in a cell, tissue, organ, animal or patient, including, but not limited to, at least one of: acute or chronic bacterial infection, acute and chronic parasitic or infectious processes, including bacterial, viral and fungal infections, HIV infection/HIV neuropathy, meningitis, hepatitis (A, B or C, or the like), septic arthritis, peritonitis, pneumonia, epiglottitis, E. coli infection, hemolytic uremic syndrome/thrombolytic thrombocytopenic purpura, malaria, dengue hemorrhagic fever, leishmaniasis, leprosy, toxic shock syndrome, streptococcal myositis, gas gangrene, mycobacterium tuberculosis, mycobacterium avium intracellulare, pneumocystis carinii pneumonia, pelvic inflammatory disease, orchitis/epidydimitis, legionella, lyme disease, influenza a, epstein-barr virus, vital-associated hemaphagocytic syndrome, vital encephalitis/aseptic meningitis, and the like.

The present invention also provides methods for modulating or treating at least one malignant disease in a cell, tissue, organ, animal or patient, including, but not limited to, at least one of: leukemia, acute leukemia, acute lymphoblastic leukemia (ALL), B-cell, T-cell or FAB ALL, acute myeloid leukemia (AML), chromic myelocytic leukemia (CML), chronic lymphocytic leukemia (CLL), hairy cell leukemia, myelodyplastic syndrome (MDS), a lymphoma, Hodgkin's disease, a malignamt lymphoma, non-hodgkin's lymphoma, Burkitt's lymphoma, multiple myeloma, Kaposi's sarcoma, colorectal carcinoma, pancreatic carcinoma, nasopharyngeal carcinoma, malignant histiocytosis, paraneoplastic syndrome/hypercalcemia of malignancy, solid tumors, adenocarcinomas, sarcomas, malignant melanoma, hemangioma, metastatic disease, cancer related bone resorption, cancer related bone pain, and the like.

The present invention also provides methods that use TNFα antagonist engineered polypeptides or polypeptide complexes for modulating or treating at least one neurologic disease in a cell, tissue, organ, animal or patient, including, but not limited to, at least one of: neurodegenerative diseases, multiple sclerosis, migraine headache, AIDS dementia complex, demyelinating diseases, such as multiple sclerosis and acute transverse myelitis; extrapyramidal and cerebellar disorders' such as lesions of the corticospinal system; disorders of the basal ganglia or cerebellar disorders; hyperkinetic movement disorders such as Huntington's Chorea and senile chorea; drug-induced movement disorders, such as those induced by drugs which block CNS dopamine receptors; hypokinetic movement disorders, such as Parkinson's disease; Progressive supranucleo Palsy; structural lesions of the cerebellum; spinocerebellar degenerations, such as spinal ataxia, Friedreich's ataxia, cerebellar cortical degenerations, multiple systems degenerations (Mencel, Dejerine-Thomas, Shi-Drager, and Machado-Joseph); systemic disorders (Refsum's disease, abetalipoprotemia, ataxia, telangiectasia, and mitochondrial multi.system disorder); demyelinating core disorders, such as multiple sclerosis, acute transverse myelitis; and disorders of the motor unit' such as neurogenic muscular atrophies (anterior horn cell degeneration, such as amyotrophic lateral sclerosis, infantile spinal muscular atrophy and juvenile spinal muscular atrophy); Alzheimer's disease; Down's Syndrome in middle age; Diffuse Lewy body disease; Senile Dementia of Lewy body type; Wernicke-Korsakoff syndrome; chronic alcoholism; Creutzfeldt-Jakob disease; Subacute sclerosing panencephalitis, Hallerrorden-Spatz disease; and Dementia pugilistica, and the like.

The TNFα antagonist engineered polypeptides or polypeptide complexes can be administered before, concurrently, and/or after (referred to herein as “concomitantly with”) other drugs, such as at least one selected from an antirheumatic (e.g., methotrexate, auranofin, aurothioglucose, azathioprine, etanercept, gold sodium thiomalate, hydroxychloroquine sulfate, leflunomide, sulfasalzine), a muscle relaxant, a narcotic, a non-steroid anti-inflammatory drug (NSAID), an analgesic, an anesthetic, a sedative, a local anethetic, a neuromuscular blocker, an antimicrobial (e.g., aminoglycoside, an antifungal, an antiparasitic, an antiviral, a carbapenem, cephalosporin, a flurorquinolone, a macrolide, a penicillin, a sulfonamide, a tetracycline, another antimicrobial), an antipsoriatic, a corticosteriod, an anabolic steroid, a diabetes related agent, a mineral, a nutritional, a thyroid agent, a vitamin, a calcium related hormone, an antidiarrheal, an antitussive, an antiemetic, an antiulcer, a laxative, an anticoagulant, an erythropieitin (e.g., epoetin alpha), a filgrastim (e.g., G-CSF, Neupogen), a sargramostim (GM-CSF, Leukine), an immunization, an immunoglobulin, an immunosuppressive (e.g., basiliximab, cyclosporine, daclizumab), a growth hormone, a hormone replacement drug, an estrogen receptor modulator, a mydriatic, a cycloplegic, an alkylating agent, an antimetabolite, a mitotic inhibitor, a radiopharmaceutical, an antidepressant, antimanic agent, an antipsychotic, an anxiolytic, a hypnotic, a sympathomimetic, a stimulant, donepezil, tacrine, an asthma medication, a beta agonist, an inhaled steroid, a leukotriene inhibitor, a methylxanthine, a cromolyn, an epinephrine or analog, domase alpha (Pulmozyme), a cytokine or a cytokine antagonist.

The subject engineered polypeptides or polypeptide complexes can also be administered concomitantly with compounds that prevent and/or inhibit TNF receptor signaling, such as mitogen activated protein (MAP) kinase inhibitors; compounds which block and/or inhibit membrane TNF cleavage, such as metalloproteinase inhibitors; compounds which block and/or inhibit TNF activity, such as angiotensin converting enzyme (ACE) inhibitors (e.g., captopril); and compounds which block and/or inhibit TNF production and/or synthesis, such as MAP kinase inhibitors.

(ii) IL-1b Antagonists

In certain embodiments, the subject engineered polypeptide or polypeptide complex is an Interleukin-1 antagonist, e.g., an “IL-1 antagonist engineered polypeptide or polypeptide complex”. Interleukin-1 is a multi-functional proinflammatory cytokine that mediates innate and adaptive immune responses in multiple cell types. It is believed to play a role in numerous diseases including arthritis, asthma/allergy, osteoporosis, and stroke (for review, see Dinarello (1998) Int. Rev. Immunol. 16, 457-499). The IL-1 family actually consists of two proteins with similar biological activity, IL-1α and IL-1β as well as a nonsignaling ligand termed the IL-1 receptor antagonist (IL-1ra). All three proteins exhibit a similar tertiary structure comprised of 12β strands that make up a barrel-shaped β-trefoil with pseudo-3-fold symmetry. IL-1β is thought to be the primary circulating cytokine that mediates the systemic effects of IL-1.

IL-1 exerts its biological action by binding and activating the membrane-associated IL1R-I. A second receptor, termed the IL-1R accessory protein (AcP), is not involved in direct ligand binding but is required for IL-1 signal transduction by complexing with IL-1 and the IL1R-I. IL1R-I and AcP both contain extracellular portions with three Ig-like domains and cytoplasmic portions containing conserved signaling motifs. A third IL-1 receptor exists termed the type II IL-1R (IL1R-II) that has a extracellular structure similar to that of IL1R-I and AcP but that contains a truncated cytoplasmic tail incapable of signaling. This receptor acts as a decoy by binding IL-1 with high affinity and neutralizing its activity. IL1R-II can also be proteolytically cleaved, which releases the extracellular domain from the cell surface. This creates a soluble form of the receptor (sIL1R-II) that possesses high affinity for IL-1β, but only low affinity for IL-1α, and virtually no affinity for IL-1ra.

In certain preferred embodiments, the subject engineered polypeptides or polypeptide complexes are IL-1 antagonist engineered polypeptide or polypeptide complex that act on IL-1, particularly IL-1β, present inbiological fluids. Exemplary recipients that be adapted for use in such engineered polypeptides or polypeptide complexes include, but are not limited to, the extracellular domains of IL-1 receptors or appropriate portions thereof, IL1R-II or a portion thereof, anti-IL-1 antibodies or antigen binding fragments thereof, or peptides or small molecules that (selectively) bind IL-1.

In certain preferred embodiments, the recipient is derived from IL1R-II, e.g., a portion sufficient to specifically bind to I1-1β. For instance, the recipient can include a ligand binding domain from IL1R-II from the human IL1R-II protein (GI Accession 640248, PRI Accession 2IRT_A).

The inhibitory activity of an IL-1 antagonist engineered polypeptide or polypeptide complex can be assayed using any of a variety of cell-based and cell-free assay systems well known in the art. For instance, IL-1 antagonist engineered polypeptides or polypeptide complexes can be identified using the mixed lymphocyte response (MLR) and phytohemagglutinin A (PHA) assay, which is useful for identifying immune suppressive molecules in vitro that can be used for treating graft-versus-host disease. The results obtained from these assays are generally predictive of their in vivo effectiveness.

Another assay that be used to assess the engineered polypeptide or polypeptide complex is with respect to inhibition of immune responsiveness involves the mitogenic stimulation of lymphocytes with mitogenic substances of plant origin. The most widely used plant molecule is PHA. Although PHA stimulates DNA synthesis non-specifically in a large number of lymphocytes, unlike true antigenic stimulation which causes mitogenesis of sub-populations of lymphocytes, the susceptibility of a patient's lymphocytes to PHA stimulation has been shown to correlate with the overall immune responsiveness of the patient.

Thus, it will be appreciated as to both the mixed lymphocyte and PHA assay that they are valuable for identifying immune suppressive IL-1 antagonist engineered polypeptides or polypeptide complexes.

In addition to the above immunosuppressive assays, a secondary mixed lymphocyte reaction assay may also be used. The secondary mixed lymphocyte assays differs from the primary mixed lymphocyte reaction assays in that they employ many more primed responder cells that are responsive to the primary stimulating cells. The presence of such responsive cells is a reflection of immunological memory in an ongoing immunological response. The protocol for carrying out a secondary mixed lymphocyte assay involves performing a primary lymphocyte assay as described above, and recovering viable cells about 9-10 days after the primary mixed lymphocyte reaction exhibits little or no cell proliferation. Generally between 10% to 50% of the original input cells are recovered in viable condition. These cells are then used in the secondary mixed lymphocyte reaction.

The subject engineered polypeptides or polypeptide complexes can also be assessed for their ability to block IL-1 mediated cytokine production. Assays for cytokine production and/or proliferation of spleen cells, lymph node cells or thymocytes are well known in the art.

In still other embodiments, the subject engineered polypeptides or polypeptide complexes can be assessed for their effect on proliferation and differentiation of hematopoietic and lymphopoietic cells.

The IL-1 mediated diseases which may be treated or prevented by the IL-1 antagonist engineered polypeptides or polypeptide complexes of this invention include, but are not limited to, inflammatory diseases, autoimmune diseases, proliferative disorders, infectious diseases, and degenerative diseases. The apoptosis-mediated diseases which may be treated or prevented by the IL-1 antagonist engineered polypeptides or polypeptide complexes of this invention include degenerative diseases.

Inflammatory diseases which may be treated or prevented include, but are not limited to osteoarthritis, acute pancreatitis, chronic pancreatitis, asthma, and adult respiratory distress syndrome. Preferably the inflammatory disease is osteoarthritis or acute pancreatitis.

Autoimmune diseases which may be treated or prevented include, but are not limited to, glomeralonephritis, rheumatoid arthritis, systemic lupus erythematosus, scleroderma, chronic thyroiditis, Graves' disease, autoimmune gastritis, insulin-dependent diabetes mellitus (Type I), autoimmune hemolytic anemia, autoimmune neutropenia, thrombocytopenia, chronic active hepatitis, myasthenia gravis, multiple sclerosis, inflammatory bowel disease, Crohn's disease, psoriasis, and graft vs. host disease. Preferably the autoimmune disease is rheumatoid arthritis, inflammatory bowel disease, Crohn's disease, or psoriasis,

Destructive bone disorders which may be treated or prevented include, but are not limited to, osteoporosis and multiple myeloma-related bone disorder.

Proliferative diseases which may be treated or prevented include, but are not limited to, acute myelogenous leukemia, chronic myelogenous leukemia, metastatic melanoma, Kaposi's sarcoma, and multiple myeloma.

Infectious diseases which may be treated or prevented include, but are not limited to, sepsis, septic shock, and Shigellosis.

The IL-1-mediated degenerative or necrotic diseases which may be treated or prevented by the IL-1 antagonist engineered polypeptides or polypeptide complexes of this invention include, but are not limited to, Alzheimer's disease, Parkinson's disease, cerebral ischemia, and myocardial ischemia. Preferably, the degenerative disease is Alzheimer's disease.

The apoptosis-mediated degenerative diseases which may be treated or prevented by the IL-1 antagonist engineered polypeptides or polypeptide complexes of this invention include, but are not limited to, Alzheimer's disease, Parkinson's disease, cerebral ischemia, myocardial ischemia, spinal muscular atrophy, multiple sclerosis, AIDS-related encephalitis, HIV-related encephalitis, aging, alopecia, and neurological damage due to stroke.

(e) Biomolecular Targets in Non-Therapeutic Contexts

Engineered polypeptides or polypeptide complexes may be used in a number of non-medical applications, including but are not limited to, agriculture, environmental protection, food etc., and such engineered polypeptides or polypeptide complexes will be targeted accordingly.

Engineered polypeptides or polypeptide complexes may be used to upgrade nutritional quality and removing anti-nutritional factors from feed components, such as barley- and wheat-based feeds. Targets for such engineered polypeptides or polypeptide complexes may include gluten meal, fiber, prions (e.g., PrP, the causative agent for bovine spongiform encephalopathy), dioxin, pesticides, herbicides, starches, lipids, cellulose, pectin, certain sugars (e.g., lactose, maltose) and polysaccharides.

Engineered polypeptide or polypeptide complex may be used in industrial processes such as waste processing, textile manufacture or paper production, or essentially any other process that employs an enzyme, where the enzyme can be replaced by an engineered polypeptide or polypeptide complex with improved effectiveness. Examples of targets for such applications include cellulose, hemicellulose, pectin, lignin, starch, peroxides, phosphates and nitrates.

Engineered polypeptide or polypeptide complex may be used in detergents or other cleaning agents, providing targeted elimination of selected soils or stains. Targets for such engineered polypeptides or polypeptide complexes may include chlorophyll, hemoglobin, heme groups, hydrocarbons, avidin, ovalbumin, and various pigments and dyes.

Engineered polypeptides or polypeptide complexes may be used for the cleanup of various environmental contaminants, such oil, pesticides, herbicides and waste products from chemical manufacture. Targets for such engineered polypeptides or polypeptide complexes include hydrocarbons, halogenated hydrocarbons (particularly halogenated hydrocarbons containing aromatic moieties), cyanides, carbon monoxide, nitrous oxides, heavy metals, organometallic compounds, organophosphates and carbamates.

The invention is further illustrated by the following examples, which should not be construed as further limiting. The contents of all references, pending patent applications and published patents, cited throughout this application, as well as the Figures and Appendices X1-X4, are hereby expressly incorporated by reference.

### EXAMPLES

### Example 1

### Determination of Spatially Conserved Residue Geometry in Serine Protease Family of Polypeptides

The following procedure was used to determine the consensus geometry of the active site serine, histidine, and aspartate amino acids in serine proteases.

1. All known serine protease structures were obtained from the protein data bank (PDB) (704 PDB entries).

2. If a structural entry contained multiple positional models for a given atom, all but the first entry were ignored.

3. The subset of the structures obtained in step 1 for which the catalytic active site is known and contains one serine, histidine, and aspartate amino acid residue was selected (409 catalytic triads).

4. For each of the structures obtained in step 2, the following six distance metrics were computed:

a. The distance between the serine (Ser) alpha carbon (CA) and the histidine (His) alpha carbon

b. The distance between the serine alpha carbon and the aspartate (Asp) alpha carbon

c. The distance between the aspartate alpha carbon and the histidine alpha carbon

d. The distance between the serine beta carbon (CB) and the histidine beta carbon

e. The distance between the serine beta carbon and the aspartate beta carbon

f. The distance between the aspartate beta carbon and the histidine beta carbon

5. For each of the six distance metrics computed in step 3, the minimum, maximum, average, and standard deviation of the values of the individual metrics observed across all structures obtained in step 2 were calculated. The results of this calculation for serine proteases is set forth in Table VII:

TABLE VII

| Distance Metric | Min (Å) | Max (Å) | Avg (Å) | Std Dev (Å) |
|---|---|---|---|---|
| Ser - His CA | 7.84 | 9.24 | 8.35 | 0.19 |
| His - Asp CA | 4.62 | 7.90 | 6.50 | 0.29 |
| Ser - Asp CA | 9.21 | 10.80 | 10.13 | 0.25 |
| Ser - His CB | 6.07 | 7.54 | 6.70 | 0.20 |
| His - Asp CB | 4.74 | 6.43 | 5.74 | 0.22 |
| Ser - Asp CB | 7.76 | 9.61 | 8.72 | 0.35 |

### Example 2

### Analysis of Potential Molecules for Substitution of Spatially Conserved Motif

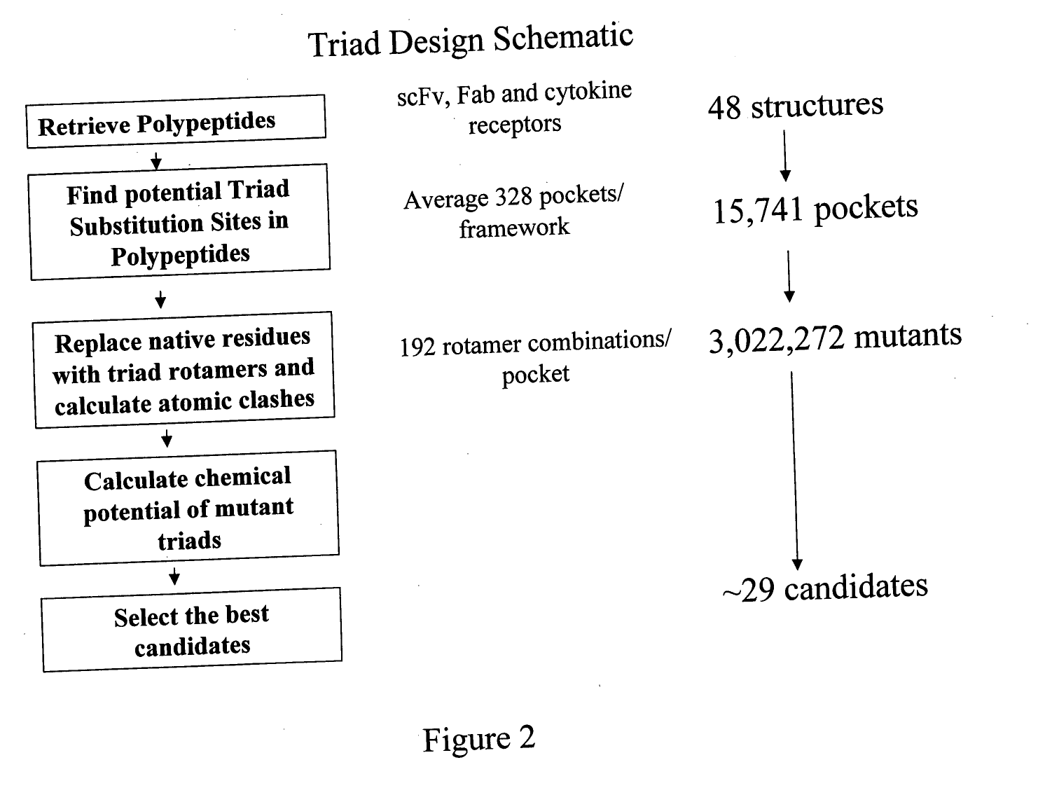

Forty eight potential molecules were analyzed as potential molecules for insertion of a serine protease triad spatially conserved motif (see FIG. 2). They include scFv antibodies (1A14, 1AP2, 1H80, 1H8N, 1LMK, 1NQB, 1DZB, 1NMC, 1FO0), IgX antibodies (1CFV, 1CLO, 1DVF, 1FLR, 1HYX, 1KEL, 1NGQ, 1OSP, 1YEC, 2H1P, 2HRP, 1BJM, 1BRE, 1GAF, 1UCB, 1VGE, 1C12), tumor necrosis factor α (monomer, dimer, and trimer), tumor necrosis factor β (monomer, dimer, and trimer), tumor necrosis factor receptor (monomer and dimer), interleukin receptor (IL-1, IL-3, and IL3-IL-5), DR5, EPO receptor, FGF receptor, GCSF receptor, IgE receptor and cyclophilin. These 48 structures produced 15, 741 potential sites of insertion for the three residues that comprise the serine protease triad. Including all possible rotamers of the substituted molecule there were 3,033,272 potential rotamers. Each of the 3,033,272 rotamers was further screened for the presence of steric clashes and the presence of the overall spatial orientation characteristic of serine proteases found in nature. Applying these two tests resulted in a subset of 29 mutants that have the correct spatial orientation characteristic of serine proteases and do not have steric clashes.

Of the 48 potential molecules analyzed the following 17 were selected as the best: 1A14, 1AP2, 1C12, 1DOG, 1DU3, 1DZB, 1EV2, 1EXT, 1F6A, 1FO0, 1H80, 1LMK, 1NMC, 1NQB, TNF-α dimer, TNF-β dimer, and the TNF-α trimer.

### Example 3

### Selection and Construction of a TNF-α Trimer with a Substituted Serine Protease Triad that Cleaves a TNF Monomer

A trimeric complex of TNF-alpha (PDB code 1TNF) was analyzed as a potential oligomeric polypeptide lacking the spatially conserved motif into which a set of residues comprising a serine protease triad could be substituted. Scanning this structure resulted in 463 possible serine protease triad insertion sites. Generation of the corresponding 88,896 rotamers of these insertions and examining of the resulting rotamers for the correct serine protease geometry and lack of steric clashes resulted in a final set of 17 mutations with the potential to introduce serine protease activity into TNF-alpha.

The resulting mutations were further screened for proximity to the proteinaceous substrate, a TNF-alpha. There was one mutation Yb119D, Yc119H, Yc59S that was proximal to a TNF monomer.

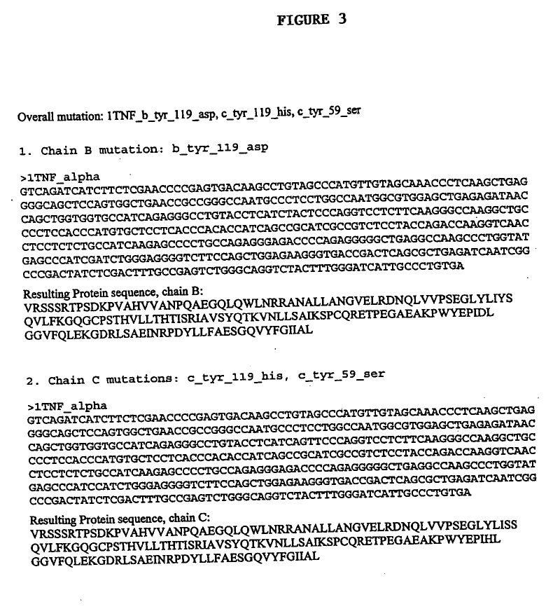

Sequence of the polypeptide chains to generate this mutant are set forth in FIG. 3.

### Example 4

### Selection and Construction of a TNF-α Trimer with a Substituted Serine Protease Triad that Cleaves a TNFr

A trimeric complex of TNF-alpha (PDB code 1TNF) was analyzed as a potential polypeptide lacking the spatially conserved motif into which a set of residues comprising a serine protease triad could be substituted. Scanning this structure resulted in 463 possible serine protease triad insertion sites. Generation of the corresponding 88,896 rotamers of these insertions and examination of the resulting rotamers for the correct serine protease geometry and lack of steric clashes resulted in a final set of 17 mutations with the potential to introduce serine protease activity into TNF-alpha.

The resulting mutations were further screened for proximity to the proteinaceous substrate, TNFr. There was one mutation Fb124D, Yc151S that was proximal to TNFr (this mutation utilizes the histidine already present at position 15 on chain C).

Sequence of this mutant may be readily deduced from the sequence of TNF-alpha.

### Example 5

### Identification of Proteases that Reduce TNF-α Activity

A survey of the literature and public domain databases (MEROPS: http://www.merops.sanger.ac.uk) for proteases that are commercially available, expressible as zymogens, and expected to cleave and inactivate TNFα [1-6] led to the selection of twenty candidate proteases, which were then tested for inactivation of TNFα using a TNF cytotoxicity assay. (See, e.g., Calkins C C, P. K., Potempa J, Travis J., Inactivation of tumor necrosis factor-alpha by proteinases (gingipains) from the periodontal pathogen, Porphyromonas gingivalis. Implications of immune evasion. J Biol Chem, 1998. 273(12): p. 6611-4; Nakamura K, K. M., Proteolysis of human tumor necrosis factor (TNF) by endo- and exopeptidases: process of proteolysis and formation of active fragments. Biol Pharm Bull:, 1996. 19(5): p. 672-7; Narhi L O, R. M., Hunt P, Arakawa T., The limited proteolysis of tumor necrosis factor-alpha. J Protein Chem, 1989. 8(5): p. 669-77; Kim Y J, C. S., Kim J S, Shin N K, Jeong W, Shin H C, Oh B H, Hahn J H., Determination of the limited trypsinolysis pathways of tumor necrosis factor-alpha and its mutant by electrospray ionization mass spectrometry. Anal Biochem., 1999. 267(2): p. 279-86; Magni F, C. F., Marazzini L, Colombo R, Sacchi A, Corti A, Kienle M G., Biotinylation sites of tumor necrosis factor-alpha determined by liquid chromatography-mass spectrometry. Anal Biochem., 2001. 298(2): p. 181-8; van Kessel K P, v. S. J., Verhoef J., Inactivation of recombinant human tumor necrosis factor-alpha by proteolytic enzymes released from stimulated human neutrophils. J Immunol., 1991. 147(11): p. 3862-8). Specifically, TNF activation of functional TNFα receptor TNFR-1 leads to apoptotic cell death, which can be quantified in a cell-based assay. (See, e.g., Idriss, H. T. N., James H., TNFα and the TNF Receptor Superfamily: Structure-Function Relationship(s). Microscopy Research and Technique, 2000: p. 184-195. 25; Locksley R M, K. N., Lenardo M J., The TNF and TNF receptor superfamilies: integrating mammalian biology. Cell, 2001. 104(4): p. 487-501; Humphreys, D. T. and M. R. Wilson, Modes of L929 cell death induced by TNF-alpha and other cytotoxic agents. Cytokine, 1999. 11(10): p. 773-82. This assay served as the basis to screen the 20 proteases for inactivation of TNFα bioactivity (see below, Table 1).

Specifically, L929 mouse connective tissue fibroblasts (ATCC catalog # CCL-1) were used to bioassay cell death induced by TNFα with the CellTiter 96® AQueous One Solution Cell Proliferation Assay system from Promega (Madison, Wis.). This system provides a colorimetric assay method for determining the number of viable cells. Briefly, for each test protease, a solution of 5 μM TNFα was digested overnight at 37° C., then bioactivity was determined for eight serial dilutions of the digestion solution. Data are mean values of triplicate determinations at each dilution of TNFα. Results from the tests on all twenty proteases are summarized in Table 1.

More specifically, 10,000 L929 cells per well were seeded in 96 well plates and cultured in DMEM+10% FBS overnight in a humidified C02 incubator. Actinomycin D was added to all wells (final concentration 1 μg/mL) and a standard TNFα survival curve was generated by adding human TNFα (RDI, Flanders, N.J.) to achieve final concentrations in the wells ranging from 100 pg/ml-1 μg/ml. Protease digestion samples of TNFα were similarly diluted and added to parallel rows of wells. Triplicate determinations were done for each dilution of TNFα. Following an overnight incubation in a humidified CO₂ incubator 20 μl of pre-mixed MTS/PES was added to each well and incubation continued for 2-4 hours at 37° C. Metabolically active viable cells reduced the assay reagent (MTS/PES includes a tetrazolium compound) into a formazan product that was soluble in tissue culture media. Absorbance was read at 490 nm in a plate reader after 4 hr to determine the number of viable cells. Complete details of the protocol were provided in Promega Technical Bulletin No. 245.

TABLE 1
Proteases tested for inactivation of TNFα.

| | | Proteases that did | |
|---|---|---|---|
| Proteases that inactivated TNFα | | not inactivate TNF | |
| MT1-MMP (0.86) | Furin | Urokinase | Plasmin |
| MMP12 (0.65) | Cathepsin G | Enterokinase | Kallikrein5 |
| Tryptase (0.62) | HIV Protease | TACE | ADAMTS4 |
| MT2-MMP (0.5) | ADAM10 | MMP3 | MT5-MMP |
| ELASTASE (1.45) | | | |
| MMP7 (1.22) | | | |
| CHYMOTRYPSIN (2.74) | | | |
| TRYPSIN (2.3) | | | |
| TNFα was digested with test proteases in overnight incubations at 37° C., then analyzed for bioactivity as described in FIG. 10. Twelve proteases had no activity | | | |
| # against TNFα; eight had varying levels of activity. Numbers in parentheses reflect log reduction in TNFα activity calculated at the 50% survival level from inactivation curves. | | | |

The survival curve for standard TNFα shows a steep reduction in survival from 100 pg/ml to 10 ng/ml. In the presence of ˜600 pg/ml TNFα reference standard only 10% of the cells survive. This is in contrast to 40% and 70% survival for the equivalent dilution of TNFα digested with MMP7 or trypsin, respectively. The curve for dilutions of trypsin-digested TNFα showed a consistent shift to the right, indicating that the bioactivity of TNFα was reduced more than two logs compared to the TNFα reference standard. Similar studies were done with all of the enzymes listed in Table 2, including MMP7. Chymotrypsin was the most active protease against TNFα (2.74 log reduction in TNFα bioactivity). Conserved protease domains from any of these enzymes are candidates for the conserved motif to be engineered into a polypeptide or polypeptide complex that binds TNFα.

### Example 6

### TNFα Binding Proteins

In addition to those described above, Applicants have generated and identified a variety of desirable TNFα binding proteins into which a catalytic domain may be engineered. The TNFR-1 p55 extracellular domain and a single chain antibody to TNFα obtained from Genetastix (San Jose, Calif.) or generated in house from standard display technologies may be used. In either instance, the protein is expected to bind TNFα with a nanomolar dissociation constant.

The sp55 domains were constructed from the full-length human ectodomain of TNFR-1, and its binding to TNFα was characterized. Briefly, human TNFR-1 encoded by the CD120A gene (accession no. NM_{—}001065; IMAGE clone 4131360, Invitrogen, Carlsbad, Calif.) was used as the template to amplify residues 30-211 in the TNFR-1 ecto-domain (protein accession no. P19438) to construct a full-length sp55. Alternative recipient polypeptides that might be evaluated may include subdomains of sTNFR-1, such as sp55Δ4 (residues 22-167) or sp55 domain 2.6 (residues 41-150). (Marsters S A, F. A., Simpson N J, Fendly B M, Ashkenazi A., Identification of cysteine-rich domains of the type 1 tumor necrosis factor receptor involved in ligand binding. J Biol Chem., 1992. 267(9): p. 5747-50; Chen P C, D. G., Chen M J., Mapping the domain(s) critical for the binding of human tumor necrosis factor-alpha to its two receptors. J Biol Chem., 1995. 270(6): p. 2874-8; Rosenberg J J, M. S., Seely J E, Kinstler O, Gaines G C, Fukuzuka K, Rose J, Kohno T, Boyle W J, Nelson A, Kieft G L, Marshall W S, Feige U, Gasser J, St Clair J, Frazier J, Abouhainze A, Moldawer L L, Edwards CK 3rd., Development of a novel, nonimmunogenic, soluble human TNF receptor type I (sTNFR-I) construct in the baboon. J Appl Physiol., 2001. 91(5): p. 2213-23). sp55 binding to TNFα was quantified using an indirect ELISA format to validate binding.

Briefly, binding domains were expressed transiently in 293T cells and captured on Ni—NTA coated wells. Binding to TNFα was quantified using the S-Tag™ system (Novagen, Madison, Wis.). The S-Tag™ system is a protein tagging and detection system based on the interaction of the 15 amino acid S-Tag peptide with ribonuclease S-protein, which is conjugated with horseradish peroxidase (HRP). Applicants constructed, expressed and purified a human TNFα fusion protein that included an N-terminal S-Tag™, then used this reagent (S-TNF) to quantify binding activity of the sp55 domains. More specifically, conditioned media, harvested and clarified by centrifugation, was diluted 1:10 into buffer (0.5 % BSA Fraction V, 0.05% Tween-20 in 1×PBS pH 7.4). Expressed proteins were captured on Ni—NTA coated wells (HisSorb plates, Catalog # 35061, Qiagen) for 1 h at room temperature with shaking and washed four times in 0.05% Tween-20 in 1×PBS to remove un-bound materials. Binding to TNFα was determined by adding 100 μL of S-TNF (or control TNFα) at 1 μg/mL in assay buffer per well, followed by incubation for 1 hr at room temperature with shaking. Plates were washed 4 times in 0.05 % Tween-20 in 1×PBS, then S-protein HRP (1:2000 in assay buffer at 100 μL/well, Novagen, Madison, Wis.) was added and incubated for 1 hr further at room temperature with shaking. A final wash step in 0.05% Tween-20 in 1×PBS was done 4 times to remove the S-protein-HRP, then 100 μL HRP substrate tetramethylbenzidine (TMB; Sigma T 4444, St. Louis, Mo.) was added per well. Color was allowed to develop for 5-45 minutes, then absorbance read at 370 nm in a Spectromax plate reader (Molecular Devices).

These experiments showed a three-fold elevation in S-TNF binding compared to non-specific binding in control samples. Binding appeared to saturate at 6-12% of conditioned media in the assay, and the dilution series showed that binding was proportional to the amount of expressed sp55 added. These results showed that the expressed sp55 domain can bind TNFα and may be used as a recipient for a catalytic domain that modulates TNFα.

As an alternative to using sp55 as an binding protein, an anti-TNFα scFV antibody may be selected from a set of eighteen that were obtained from Genetastix (San Jose, Calif.). These scFV antibodies were identified by Genetastix through use of proprietary technology (www.genetastix.com) as having TNFα binding activity. Briefly, a human scFv cDNA library was produced from polyA RNA of human spleen, lymph nodes and peripheral blood lymphocytes through amplification of V_{H} and V_{L} sequences that were assembled in frame with a GAL4 activation domain (AD). The 18 scFvs were identified as binding human TNFα-lexA DNA binding domain when co-expressed intracellularly in yeast. The Genetastix scFvs expression vectors were obtained in the form of bacterial periplasmic expression vector pET25B (Novagen, Madison, Wis.). Standard recombinant DNA methods were used to subclone the scFv coding sequences into the pSecTag2A vector. The constructs were then sequenced to verify the structures. These scFv anti-TNFα antibodies is expressed and purified as described for other proteins, above, then analyzed for binding to TNFα. An indirect ELISA is used for TNFα based on the S-Tag™ system (see above) to identify scFvs that show high affinity binding to TNFα for use as a recipient polypeptide. Further quantitative determinations of binding affinities for TNFα may be performed.

Incorporation by Reference

All publications and patents mentioned herein are hereby incorporated by reference in their entirety as if each individual publication or patent was specifically and individually indicated to be incorporated by reference. In case of conflict, the present application, including any definitions herein, will control.

Equivalents

While specific embodiments of the subject inventions are explicitly disclosed herein, the above specification is illustrative and not restrictive. Many variations of the inventions will become apparent to those skilled in the art upon review of this specification and the claims below. The full scope of the inventions should be determined by reference to the claims, along with their full scope of equivalents, and the specification, along with such variations.

What is claimed is:

1. A method for engineering a spatially conserved catalytic motif into a recipient polypeptide that binds a target, the method comprising:
a) obtaining a spatial relationship for the amino acid residues of a spatially conserved catalytic motif;
b) identifying a set of amino acid residues in the recipient polypeptide, wherein said set of residues have a geometric relationship that matches the spatially conserved geometry of the catalytic motif, and wherein the recipient polypeptide binds to a target that is an extracellular signaling molecule with a K_{D} of 10⁻⁶M or less; and
c) substituting said set of residues in said recipient polypeptide with a set of amino acid residues making up said catalytic motif;
thereby engineering a catalytic motif into a recipient polypeptide that binds to an extracellular signaling molecule.

2. A method for engineering a spatially conserved target binding motif into a recipient polypeptide that catalytically modifies a target, the method comprising:
a) obtaining a spatial relationship for the amino acid residues of a spatially conserved target binding motif, wherein the target binding motif mediates binding to a target that is an extracellular signaling molecule with a K_{D} of 10⁻⁶M or less; and
b) identifying a set of amino acid residues in a recipient polypeptide, wherein said set of residues have a geometric relationship that matches the spatially conserved geometry of the target binding motif; and
c) substituting said set of residues in said recipient polypeptide with a set of amino acid residues making up said target binding motif;
thereby engineering a target binding motif that binds to an extracellular signaling molecule target into a recipient polypeptide that catalytically modifies the target.

3. The method of claim 1, wherein the targeted extracellular signaling molecule is an inflammatory cytokine.

4. The method of claim 1, wherein the targeted extracellular signaling molecule is TNF-α.

5. The method of claim 1, wherein the recipient polypeptide is selected from among: a binding portion of an anti-TNF-α antibody, a soluble ligand binding portion of a TNF-α receptor and a TNF-α polypeptide.

6. The method of claim 1, wherein the catalytic motif catalyzes proteolysis of the target.

7. The method of claim 1, wherein the catalytic motif comprises a serine protease triad.

8. The method of claim 1, wherein said set of residues identified in said recipient polypeptide are less than about 10 Å away from a target binding site.

9. The method of claim 1, further comprising constructing a model of said recipient polypeptide containing said set of substituted residues in silico and determining the existence of atomic clashes between atoms in said model.

10. The method of claim 9, wherein the model is rejected if atomic clashes are present between atoms in said model.

11. The method of claim 1, further comprising constructing a model of said recipient polypeptide containing said set of substituted residues in silico and comparing the polypeptide backbone of said recipient polypeptide in the presence and absence of said set of substituted residues.

12. The method of claim 11, further comprising determining the root mean squared deviation of α-carbon atoms in said polypeptide backbone in the presence and absence of said set of substituted residues.

13. The method of claim 12, wherein the model is rejected if there is a root mean squared deviation of greater than 2 Å between backbone α-carbon atoms.

14. The method of claim 1, wherein said spatially conserved motif comprises a number of amino acid residues selected from the group consisting of 2, 3, 4, 5, 6, 7, and 8.

15. The method of claim 1, wherein identifying said set of amino acid residues in the recipient polypeptide includes modeling the presence of a β-carbon on a glycine residue of the recipient polypeptide.

16. The method of claim 15, wherein said set of residues in said recipient polypeptide comprise a glycine residue.

17. A method for engineering a spatially conserved catalytic motif into a recipient polypeptide that binds a target, the method comprising:
d) obtaining a spatial relationship for the amino acid residues of a spatially conserved catalytic motif;
e) identifying a set of amino acid residues in the recipient polypeptide, wherein said set of residues have a geometric relationship that matches the spatially conserved geometry of the catalytic motif, and wherein the recipient polypeptide binds to a target that is a receptor for an extracellular signaling molecule with a K_{D} of 10⁻⁶M or less; and
f) substituting said set of residues in said recipient polypeptide with a set of amino acid residues making up said catalytic motif;
thereby engineering a catalytic motif into a recipient polypeptide that binds to a receptor for an extracellular signaling molecule.

18. The method of claim 17, wherein the target is a receptor for TNF-α.

19. The method of claim 17, wherein the recipient polypeptide is a ligand for the receptor.

20. The method of claim 18, wherein the recipient polypeptide is a TNF-α.

21. A method for engineering a spatially conserved motif into a recipient polypeptide, the method comprising:
a) obtaining a spatial relationship for the amino acid residues of a spatially conserved motif;
b) identifying a set of amino acid residues in a recipient polypeptide, wherein said set of residues have a geometric relationship that matches the spatially conserved geometry of the motif, and wherein identifying said set of amino acid residues in the recipient polypeptide includes modeling the presence of a β-carbon on a glycine residue of the recipient polypeptide; and
c) substituting said set of residues in said recipient polypeptide with a set of amino acid residues making up said motif;
thereby engineering a spatially conserved motif into a recipient polypeptide.

22. The method of claim 21, wherein the recipient polypeptide binds to a target molecule.

23. The method of claim 22, wherein the target molecule is an extracellular signaling molecule is an inflammatory cytokine.

24. The method of claim 23, wherein the extracellular signaling molecule is TNF-α.

25. The method of claim 21, wherein the motif is a catalytic motif.

26. The method of claim 25, wherein the catalytic motif catalyzes proteolysis of the target.

27. The method of claim 26, wherein the catalytic motif comprises a serine protease triad.

28. The method of claim 21, wherein said set of residues identified in said recipient polypeptide are less than about 10 Å away from a target binding site.

29. The method of claim 21, further comprising constructing a model of said recipient polypeptide containing said set of substituted residues in silico and determining the existence of atomic clashes between atoms in said model.

30. The method of claim 29, wherein the model is rejected if atomic clashes are present between atoms in said model.

31. The method of claim 21, further comprising constructing a model of said recipient polypeptide containing said set of substituted residues in silico and comparing the polypeptide backbone of said recipient polypeptide in the presence and absence of said set of substituted residues.

32. The method of claim 31, further comprising determining the root mean squared deviation of α-carbon atoms in said polypeptide backbone in the presence and absence of said set of substituted residues.

33. The method of claim 32, wherein the model is rejected if there is a root mean squared deviation of greater than 2 Å between backbone α-carbon atoms.

34. The method of claim 21, wherein said spatially conserved motif comprises a number of amino acid residues selected from the group consisting of 2, 3, 4, 5, 6, 7, and 8.

35. The method of claim 21, wherein said set of residues in said recipient polypeptide comprise a glycine residue.

36. A method for engineering a spatially conserved motif into a recipient polypeptide complex, the method comprising:
a) obtaining a spatial relationship for the amino acid residues of a spatially conserved motif;
b) identifying a set of amino acid residues in a recipient polypeptide complex, wherein said set of residues have a geometric relationship that matches the spatially conserved geometry of the motif, and wherein said set of amino acid residues includes amino acid residues of more than one polypeptide in the recipient polypeptide complex; and
c) substituting said set of residues in said recipient polypeptide complex with a set of amino acid residues making up said motif;
thereby engineering a spatially conserved motif into a recipient polypeptide complex.

37. The method of claim 36, wherein the recipient polypeptide binds to a target molecule.

38. The method of claim 37, wherein the target molecule is an extracellular signaling molecule is an inflammatory cytokine.

39. The method of claim 38, wherein the extracellular signaling molecule is TNF-α.

40. The method of claim 37, wherein the target molecule is a receptor.

41. The method of claim 40, wherein the receptor is a receptor for an extracellular signaling molecule.

42. The method of claim 41, wherein the receptor is a receptor for TNF-α.

43. The method of claim 36, wherein the motif is a catalytic motif.

44. The method of claim 43, wherein the catalytic motif catalyzes proteolysis of a target.

45. The method of claim 44, wherein the catalytic motif comprises a serine protease triad.

46. The method of claim 36, wherein the recipient polypeptide complex is an antibody or a complex comprising an antigen binding portion of an antibody.

47. The method of claim 36, wherein said set of residues identified in said recipient polypeptide complex are less than about 10 Å away from a target binding site.

48. The method of claim 36, further comprising constructing a model of said recipient polypeptide complex containing said set of substituted residues in silico and determining the existence of atomic clashes between atoms in said model.

49. The method of claim 48, wherein the model is rejected if atomic clashes are present between atoms in said model.

50. The method of claim 36, further comprising constructing a model of said recipient polypeptide complex containing said set of substituted residues in silico and comparing the polypeptide backbones of said recipient polypeptide complex in the presence and absence of said set of substituted residues.

51. The method of claim 50, further comprising determining the root mean squared deviation of α-carbon atoms in said polypeptide backbones in the presence and absence of said set of substituted residues.

52. The method of claim 36, wherein the model is rejected if there is a root mean squared deviation of greater than 2 Å between backbone α-carbon atoms.

53. The method of claim 36, wherein said spatially conserved motif comprises a number of amino acid residues selected from the group consisting of 2, 3, 4, 5, 6, 7, and 8.

54. The method of claim 36, wherein identifying said set of amino acid residues in the recipient polypeptide includes modeling the presence of a β-carbon on a glycine residue of the recipient polypeptide.

55. The method of claim 54, wherein said set of residues in said recipient polypeptide comprise a glycine residue.

56. A method for engineering a spatially conserved partial motif into a target binding recipient polypeptide or polypeptide complex, the method comprising:
a) obtaining a spatially conserved residue geometry for a spatially conserved motif;
b) identifying a set of amino acid residues in a holo-complex comprising the recipient polypeptide or polypeptide complex and the target, wherein said set of residues have a geometric relationship that matches the spatially conserved geometry of the catalytic motif and wherein at least one of said amino acid residues of the set occur in the target;
c) identifying in the set of amino acid residues a subset of amino acid residues that are present on the recipient polypeptide or polypeptide complex;
d) substituting the subset of residues in said recipient polypeptide or polypeptide complex with the corresponding amino acid residues of said motif;
thereby engineering a partial motif into a recipient polypeptide or polypeptide complex such that binding of the engineered recipient polypeptide or polypeptide complex to the target reconstitutes the motif.

57. An engineered polypeptide or polypeptide complex that binds to an extracellular signaling molecule and comprises an engineered spatially conserved catalytic motif which catalytically modifies the extracellular signaling molecule.

58. The engineered polypeptide or polypeptide complex of claim 57, wherein the catalytic motif confers protease activity.

59. The engineered polypeptide or polypeptide complex of claim 58, wherein the catalytic motif comprises a serine protease triad.

60. The engineered polypeptide or polypeptide complex of claim 56, wherein the extracellular signaling molecule is an inflammatory cytokine.

61. The engineered polypeptide or polypeptide complex of claim 60, wherein the extracellular signaling molecule is TNF-α.

62. The engineered polypeptide or polypeptide complex of claim 56, wherein the polypeptide or polypeptide complex into which the catalytic motif has been engineered is selected from among: a soluble receptor that binds the target extracellular signaling molecule, an antibody that binds the target, a portion of an antibody that binds the target and a monomer or multimer of the extracellular signaling molecule.

63. An engineered polypeptide or polypeptide complex that binds to a receptor for an extracellular signaling molecule and comprises an engineered spatially conserved catalytic motif which catalytically modifies the extracellular signaling molecule.

64. The engineered polypeptide or polypeptide complex of claim 63, wherein the catalytic motif confers protease activity.

65. The engineered polypeptide or polypeptide complex of claim 63, wherein the catalytic motif comprises a serine protease triad.

66. The engineered polypeptide or polypeptide complex of claim 63, wherein the receptor is a receptor for an inflammatory cytokine.

67. The engineered polypeptide or polypeptide complex of claim 66, wherein the receptor is a receptor for TNF-α.

68. The engineered polypeptide or polypeptide complex of claim 63, wherein the polypeptide or polypeptide complex into which the catalytic motif has been engineered is selected from among: an antibody that binds the target, a portion of an antibody that binds the target and a monomer or multimer of the extracellular signaling molecule.
